(19) United States
(12) Patent Application Publication
(10) Pub. No.: US 2026/0260716 A1
(43) Pub. Date: Sep. 3, 2026

(54) METHOD FOR DETERMINING A MEASURE FOR AN APPLICATION PERFORMANCE OF A POLYMER

(71) Applicant: BASF SE, Ludwigshafen am Rhein (DE)

(72) Inventors: Volker Settels, Ludwigshafen am Rhein (DE); Ning Wang, Ludwigshafen am Rhein (DE); Jessica Eleanor Bean, Ludwigshafen am Rhein (DE); Tobias Wagener, Ludwigshafen am Rhein (DE)

(21) Appl. No.: 18/838,412

(22) PCT Filed: Feb. 17, 2023
(86) PCT No.: PCT/EP2023/054093

§ 371 Date: Aug. 14, 2024

(30) Foreign Application Priority Data
Feb. 18, 2022 (EP) 22157567.3
Feb. 18, 2022 (EP) 22157573.1
Feb. 18, 2022 (EP) 22157580.6

(51) Int. Cl. G16C 60/00; G16C 20/10

(52) U.S. Cl. CPC G16C 60/00; G16C 20/10

(57) ABSTRACT

The invention refers to an apparatus for determining a performance measure. A providing unit (112) provides a digital representation of a target polymer indicative of characterising parameters of a polymer. A providing unit (113) provides a digital representation of application conditions indicative of application parameters defining the application conditions. A providing unit (114) provides a model based on the provided digital representation of the application conditions. The model is adapted to determine a performance measure of a polymer with respect to the application conditions. The model is a data driven model parameterised with respect to the application conditions such that it can determine a performance measure of a polymer based on the digital representation of the polymer. A determining unit (115) for determining the performance measure of the target polymer based on the provided determination model and the digital representation of the target polymer.

### FIELD OF THE INVENTION

The invention relates to a method, an apparatus and a computer program product for determining a measure for an application performance of a target polymer. Moreover, the invention refers to a method for determining a target synthesis specification indicative of a target polymer comprising a target measure for an application performance. Further, the invention refers to a training method, a training apparatus and a training computer program for training a data driven determination model utilizable by the method, apparatus and computer program product for determining the biodegradability of a polymer and/or for determining a target synthesis specification. Moreover, the invention refers to a method and apparatus for providing an interface for the determination of the target measure of a polymer.

### BACKGROUND OF THE INVENTION

Generally, polymers are widely used in industrial and/or daily use products due to their broad range of application properties. The use of polymers encompasses amongst others coatings, personal care products, washing detergents, lubricants, packaging and foams. However, this widely spread application and the nearly infinite possibility for polymers makes it often difficult to find polymers for a specific application in a sensible time without a high personnel, resource and cost intensive search, for example, for example, by synthesising a plurality of candidate polymers and measuring their application performance and then selecting a suitable polymer, if present. Further, for most applications not only one performance parameter has to be met, but a plurality of different ones. For example, not only has the polymer to be suitable for being used as washing detergent, but also has it to be biodegradable when in contact with waste water. This makes it even more difficult to find suitable polymers in early stages of a product design process. Thus, it would be advantageous to provide a possibility to predict an application performance measure of a polymer accurately and in a computationally inexpensive manner.

### SUMMARY OF THE INVENTION

It is an object of the present invention to provide a method, an apparatus and a computer program product that allow for an accurate determination of an application performance measure of a polymer that is computationally inexpensive and can robustly be applied to new polymers. Further, it is an object of the invention to provide a method, an apparatus and a computer program product that allow for an accurate and resource inexpensive determination of a synthesis specification of a polymer fulfilling a predetermined target. Moreover, it is further an object of the invention to provide a training method, a training apparatus and a computer program product that allow to provide a determination model that is usable in the method, apparatus and computer program and that can be trained to provide a good determination accuracy by utilizing less computational resources.

In a first aspect of the present invention, a computer implemented method for determining a measure for an application performance of a target polymer, wherein the method comprises a) providing a digital representation of a target polymer indicative or associated with characterising parameters of a polymer, b) providing a digital representation of application conditions for the target polymer, wherein a digital representation of the application conditions is indicative or associated with application parameters defining the application conditions, c) providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, and d) determining the application performance measure of a target polymer based on the provided determination model and the digital representation of the target polymer.

Since the determination model is specifically adapted to determine an application performance measure of a polymer under specifically provided application conditions defined by respective application parameters, the application performance measure of a polymer for the respective application can be determined very accurately. Moreover, since the determination model has specifically been trained for one or more specific application conditions, less training data becomes necessary for the training. Further, the determination model becomes more flexible with respect to determining the application performance measure of new polymers not being part of the training data set. Thus, the method allows for an accurate determination of an application performance measure that is computationally inexpensive and can be applied flexibly also to new polymers. Furthermore, currently utilized methods for measuring an application performance measure of a polymer are often extremely time consuming and can take a lot of resources, whereas the above described method allows to provide results essentially immediately without polymer material consumption. Thus, not only the technical requirements for finding a suitable polymer can be reduced, but also the time required for designing a new product for an application can be considerably shortened. Moreover, providing an easy possibility for accurately determining an application performance of a product that can be taken into account already during the design process, allows to design the product such that the real conditions in which a product is later used are also taken into account leading, for example, to a decreased waste production or an increased security.

Moreover, since the characterizing parameters are utilized that contain, for example, chemical and/or physicochemical information of the polymer, e.g. the polymer structure and composition, the training of a respective determination model can be improved. In particular, utilizing the characterizing parameters allows training of such models with less training data, because some of the correlation information that needs to be learned is already presented to the model by using the characterizing parameters. This, further allows to save tests and experiments necessary for providing the training data set.

Development of new chemical products that are tailored to application requirements is a predominant problem in modern chemical industries. Recently, often further requirements are also raised, for instance, related to the environmental impact or security of the chemical product along the life cycle of the chemical product. For example, one important aspect of the environmental impact can be the prevention of microplastics. Microplastics are an increasing problem and can be avoided if the polymeric material is biodegradable. To evaluate biodegradation currently a series of standardized tests, are used. For biodegradability, a variety of tests exists with specified conditions (e.g. ISO13432, ISO14852, ISO14855, ISO17556 and OECD 301). Such standardized tests often strike a balance between a time-efficient testing (shortest 14 days, longest 24 months) and real-life conditions. In fact, higher temperatures than real conditions are often used to speed up the testing time. Companies developing new polymers need to invest significant resources in self-assessing product sustainability or security and in certification. The overall assessment of such additional conditions, including laboratory spaces and equipment, becomes costly and time consuming.

Thus, there is a need to early identify an application performance measure of a new material in the development process. The proposed method of determining an application performance measure as disclosed herein enables a faster and more efficient way of developing new materials. In an early phase, even before synthesis of the polymer, the application performance can be determined. This allows to determine whether the polymer is suited for market entry. This leads to a faster time to market. This also allows to reduce waste production, because the polymer does not need to be synthesized to determine the application performance. The proposed method provides a digital twin of measuring the application performance measure of a polymer.

Further, the standard measurements and tests for an application performance are often time consuming, for example, include waiting times of up to several months or even years and resource intensive when performing extensive test series. In particular when developing new polymers for respective applications these time-consuming tests can strongly limit the development process. In this context the invention allows to provide results for a new polymer instantly strongly decreasing the time after which results are available.

Moreover, due to the incredibly high number of possible, often not even fully explored polymers, potentially suitable for a specific application, today a technical product engineer, given the technical task of finding a polymer that is not only suitable for a specific application, but also fulfills respective target properties, in particular, a target application performance measure, has to synthesize and test huge amounts of possible polymers, or go through huge datasets and libraries in which potential polymers are stored in order to find a respective polymer that might fit the application. Even when utilizing sophisticated design of experiment methods, still a very high number of possible polymers have to be synthesized and experimentally tested. In this context the above described method allows to assist a user, for instance, a technical product engineer, to find potentially suitable polymers automatically and much faster. In particular, by utilizing the above method the user only has to synthesize and test potentially suitable polymers for which the method has determined that it is very likely that they fulfill the respective target properties. Accordingly, unnecessary synthesis and testing of polymers can be avoided. Thus, the method allows a user to perform a technical task of finding a polymer suitable for a technical application faster and more efficient.

The method refers to a computer implemented method and thus can be performed by a general or dedicated computer adapted to perform the method, for instance, by executing a respective computer program. The method is adapted to determine, in particular, predict an application performance measure of a predetermined target polymer. An application performance measure can be any measure that defines a performance of the polymer in a specific application. Generally, such an application performance measure is an intrinsic characteristic of a polymer that is determined by the physical and chemical nature of the polymer itself. In a preferred example the performance measure is a biodegradability of a polymer. In this case, the biodegradability is a measure for the performance of the polymer to biodegrade in a respective waste environment of the intended application. For example, for a washing detergent the intended application waste environment is wastewater. Generally, a biodegradable polymer refers to a polymer that can be degraded by biological processes, in particular, a biodegradable polymer can refer to a polymer that can be assimilated by bacteria and/or fungi to give environmentally friendly products, i.e. to decompose into non-polluting residuals. For example, it is preferred that the biodegradability of the polymer refers to any one of a mineralization characteristic, a biotransformation characteristic and/or a decomposition of the polymer after a specific timeframe. The biodegradability is usable, in particular, for validating a biodegradation, i.e. a degradation characteristic, of a polymer, for example, for a specific biodegradation environment, i.e. biodegradation habitat. For example, the biodegradability can be compared with required biodegradabilities for a specific application and thus it can be determined if a respective polymer is suitable for a respective application. In another preferred embodiment, the application performance measure is an adhesive property of the polymer in a predetermined environment. An adhesive property can generally determine adhesive characteristic of a polymer with respect to adhering at least two surfaces to each other. For example, an adhesive property can be an adhesion strength, a removability, timing of the onset of adhesion or the loss of adhesion, etc. In a further preferred embodiment the application performance measure can be a solubility of the polymer in a predetermined fluid environment. For example, the solubility can then be defined by an amount of soluble polymer in a specific fluid environment, a concentration in the solution, a time until a predetermined concentration is reached, etc. In a further preferred embodiment, the application performance measure is a washing performance measure of the polymer in a predetermined environment. A washing performance can generally determine the ability of a polymer to remove a substance from an object, for instance, to remove a certain stain colour from a textile. For example, a washing performance measure can be any of an amount of substance removed from an object in washing process, a solubility of a predetermined substance, like fat, and a foaming measure. In a further preferred embodiment the application performance measure is a burning behaviour of the polymer. Generally, the burning behaviour can be defined by one or more properties that can refer to any properties that allow for a quantification of the burning behaviour of a polymer, for example, to determine when the polymer in a certain situation will burn. In particular, a burning property reflects the nature of the polymer when subjected to a heat source. Preferably, the one or more burning properties indicative for the burning behaviour of the polymer comprised at least one of a heat release rate, a peak heat release, a total heat release, an average rate of heat emission, a maximum rate of heat emission, effective heat of combustion, flame height, burning time, afterburn time, mass loss, mass loss rate, fire growth rate, total smoke production, smoke growth rate, dripping behavior, ignition time and extinguishment time.

Generally, the application performance measure can refer to only one value, for instance, to an ignition temperature or percentage of biodegradation after a predetermined time of the polymer, but can also refer to more than one value, for instance, can refer to a temperature profile or biodegradation profile within a sample made from the polymer when subjected to a predetermined application condition. Generally, the application performance measure is an intrinsic characteristic of a polymer. In this context, an intrinsic characteristic of the polymer refers to a property of the polymer that is caused by and thus reflects the nature of the polymer, i.e. the structure, composition, etc., with respect to a specific context.

Generally, the polymer can be any material made by polymerization. Preferably, the target polymer is a synthetic polymer. In an embodiment, a synthetic polymer may be a chemical compound which is produced by a chemical production from one or more starting mate-rial(s), such as monomers, and which comprises at least two monomer units. The monomer units may be regarded as subunits of the polymer. The polymer may be prepared from the monomers by polymerization reactions. The polymer may be produced from a single type of monomer or from different monomers or different monomers and other polymers. The monomer units may be distributed randomly, have a compositional drift or may be present as blocks within the polymer. The polymer may be a linear polymer. The polymer may be a branched polymer. The polymer may be a crosslinked polymer. The polymer may be soluble. The polymer may be a solid crystalline, semi-crystalline or amorphous material. The polymer may have a tertiary structure, e.g. a dispersion particle, a micelle or a foam.

In a first step, the method comprises providing a digital representation of a target polymer indicative or associated with characterising parameters of a polymer. In particular, the providing can refer to receiving the digital representation from an input of a user using, for instance, a respective input unit. Moreover, the providing can also refer to accessing a storage unit on which the digital representation is already stored. Further, the providing can also comprise receiving characterising parameters, for instance, via a network connection from other sources and providing the received characterizing parameters as digital representation. Moreover, being indicative of or associated with characterising parameters of a polymer is defined as allowing to access the information of the characterizing parameters.

For example, the digital representation can directly comprise the characterizing parameters, for example, in form of values for respective quantities. However, the digital representation can also be a link to the respective characterizing parameters via which the characterizing parameters can be accessed, or the digital representation can refer to an identifier that is associated with the characterizing parameters and allows to utilize a respective look up storage in order to access the characterizing parameters. Moreover, the digital representation can also refer to information that allows to derive the characterizing parameters using one or more known relations. For example, a synthesis specification or a structural formula of a polymer can be utilized as digital representation allowing to derive, using known chemical and physical laws and relations, respective characterizing parameters.

Generally, throughout the following description referring a parameter comprises referring both to the respective quantity and also to a specific value of the quantity if not explicitly defined otherwise. For example, a parameter being a temperature always refers to the quantity being a temperature and also to a specific value of the temperature being set for the quantity. Since in most cases the explicit value of the parameter can be different for different embodiments and application cases the value is generally not mentioned. However, providing a parameter generally means providing the quantity, e.g. the information that a value is a temperature, and also the value of the quantity itself.

Preferably, the characterizing parameters of a polymer consist of polymer physicochemical parameters. Preferably, the digital representation is indicative of and/or comprises polymer physicochemical parameters, preferably, referring to polymer descriptors, wherein the polymer physicochemical parameters are indicative of the physicochemical characteristics of the polymer. In particular, the polymer physicochemical parameters are indicative of parameters quantifying the physicochemical characteristics of the polymer. In this context, the term “physicochemical characteristics” refers to physical and/or chemical characteristics of the polymer. However, the digital representation can also be provided such that it allows to derive physicochemical characteristics, for example, in form of the polymer descriptors, for instance, by providing a representation of the polymer for which respective physicochemical characteristics are already stored or can be determined, for instance, by respective polymer descriptor calculations. Preferably, the digital representation refers to at least one of a recipe, a structural formula, a brand name, an IUPAC name, a chemical identifier and a CAS number, a SMILES name, an InCHI name of the polymer.

In a preferred embodiment, the polymer physicochemical parameters are parameters quantifying the physicochemical characteristics of subgroups of the polymer. In this embodiment, the digital representation can also be provided such that it allows to derive the polymer physicochemical parameters by determining subgroups of the polymer and to determine the polymer physicochemical parameters based on physicochemical characteristics of the determined subgroups. Generally, a subgroup refers to a part of the polymer, wherein all subgroups of a polymer together form the polymer. For example, a subgroup can refer to a part of the polymer, wherein the subgroups are linked together successively along a chain or network to form the polymer. Preferably, the subgroups of the polymer refer to repeating units that describe a part of the polymer which when repeated produces the complete polymer chain. However, in some cases, a subgroup can also refer to a single part of the polymer that is not repeated. Moreover, it is preferred that the subgroups comprise parts that are repeated, for example, a subgroup of a polymer can comprise a repeating core also present in other subgroups and further additional parts that are not present in other subgroups. Preferably, the subgroups refer to at least one of polymerized monomers or oligomer fragments. More preferably, the subgroups refer to polymerized monomers. In this context, polymerized monomers refer to monomers after their polymerization sometimes also called “mer unit” or “mer”. In particular, polymerized monomers do not refer to monomers, i.e. raw materials, as present in a reaction mixture before polymerization, but refer to repeating units derived from monomers that have been changed during or after the polymerization. Thus, subgroup descriptors determined for polymerized monomers are different from subgroup descriptors determined for unreacted monomers before polymerization. It has been found by the inventors that in particular the polymerized monomers allow to determine polymer descriptors from the subgroup descriptors of the polymerized monomers that allow for an accurate determination of the biodegradability. In a preferred embodiment, the digital representation of the polymer comprises subgroups provided as a molecular model which is indicative of its chemical structure of the subgroup after its polymerization. Even more preferably, the molecular model of a subgroup is determined in a way that is suited for cheminformatics regarding a number and type of atoms and their connectivity. Moreover, additionally or alternatively to a molecular model of a subgroup treating the subgroup as a monomer structure, also a molecular model referring to an oligomer model can be utilized that takes into account effects of neighbouring molecular structures of the subgroup in the polymer.

Generally, if the digital representation of the polymer does not directly comprise the polymer physicochemical parameters, it is preferred that the polymer physicochemical parameters are determined by determining the subgroups of the polymer. For example, respective subgroups of the polymer can be determined utilizing known methods. However, it is preferred that the determination of the subgroups of the polymer is performed in accordance with later described embodiments of the invention. In particular, it is preferred that the subgroups are determined such that between atoms of different subgroups in the polymer the bond is as least polarized as possible and, preferably, with a bond order as small as possible (e.g. a CC single bond). Additionally, it is preferred that the subgroups representing a polymer comprise the same number of active non-hydrogen-atoms then the polymer. Besides the active atoms, a subgroup can also contain further atoms, which can be ignored during computing the physical-chemical parameters of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languages like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of subgroups of the polymer are directly derivable and, for example, RDkit can be used to determine from the SMILES of the monomers the SMILES, i.e. the number and connectivity of the atoms, of the subgroups.

The determined subgroups of the polymer are associated with subgroup physicochemical parameters quantifying characterizing parameters of the subgroups in the polymer, preferably, also the subgroup physicochemical parameters refer to subgroup descriptors. In particular, it is preferred that if the polymer physicochemical parameters are not directly provided by the digital representation, the polymer physicochemical parameters are determined by determining a respective subgroup physicochemical parameter for each of the subgroups and to determine the polymer physicochemical parameters based on the subgroup physicochemical parameters of the subgroups, for instance, by averaging or summing up. Thus, the method preferably comprises first providing or determining for the polymer the subgroups from the digital representation of the polymer, then to determine or provide the subgroup physicochemical parameters, i.e. values of the parameters quantifying the physicochemical characteristics, of the subgroups, and then to determine the polymer physicochemical parameters based on the subgroup physicochemical parameters of each polymer. Alternatively, a molecular graph representation of the polymer can be used. Here, atom- and bond-resolved physicochemical parameters are used as additional information at the vertices and edges of the molecular graph representation.

Preferably, the polymer physicochemical parameters refer to polymer descriptors referring to at least one of constitutional descriptors, count descriptors, list of structural fragments, fingerprints, graph invariants, 3D-descriptors and/or higher dimensional descriptors that are indicative of parameters quantifying physicochemical characteristics of the polymer. In a preferred embodiment the polymer descriptors refer to 3D descriptors, in particular, quantum chemical descriptors. Moreover, the inventors have found that in particular a molar mass and/or the amount or type of chemical moieties is important to describe the biodegradation of a polymer very accurately. Thus, it is in particular preferred that the physicochemical parameters comprise a molar mass of the polymer. Generally, the polymer physicochemical parameters can be derived from the subgroup physicochemical parameters, thus, also the subgroup physicochemical parameters can refer to the same descriptors as stated above. However, the physicochemical parameters can also be derived without utilizing subgroups, for instance, by molecular simulations of the whole polymer. In the following the possible physicochemical parameters are defined in more detail. Also in these cases the defined physicochemical parameters can refer directly to the polymer physicochemical parameters or, optionally, to the subgroup physicochemical parameters.

A constitutional descriptor can refer to any of a potential, average molecular weight, polydispersity, charge, spin, boiling point, melting point, enthalpy of fusion, dissociation constant, Hansen parameter, protic, polar and dispersive contributions, Abraham parameter, retention index, total polar surface area, receptor binding constant, Michaelis-Menten constant, inhibitor constant, mutagenicity, LD50, bioconcentration, toxicity, biodegradation profile and viscosity.

A count descriptor can refer to any of a sum of atomic electro negativities, a sum of atomic polarizabilities, an amount of ingredients, a weight fraction of ingredients, a ratio of amounts or weight fractions of ingredients, an amount of subgroups, a weight fraction of subgroups, a ratio of amount or weight fraction of subgroups, a number of atoms and non H-atoms, a number of certain atoms, in particular, H, B, C, N, O, P, S, Si, Hal and heavy atoms, a number of H-donor and H-acceptor atoms, a number of bonds, non-H or multiple bonds, a number of double, triple and aromatic bonds, a number of functional groups or chemical motifs, a weight fraction of functional groups or chemical motifs, a ratio of functional groups or chemical motifs, a ratio of functional groups or chemical motifs and ingredients, a sum or count of bond orders, an aromatic ratio, a number of rings or circuits, a number of un-paired electrons, a number of rotatable bonds, rotatable bond fractions, and a number of conformers.

Polymer descriptors referring to a list of structural fragment descriptors can refer to at least one of a list of molecular fractions, a list of functional groups, a list of bonds, and a list of atoms. Fingerprint descriptors comprise preferably, at least one of MACCS keys, preferably, in bit format or count format, Morgan and other circular fingerprints, preferably, in bit format or count format, topological torsion, atom pairs, infrared and related spectra, Pub-Chem fingerprint, substructure fingerprint, and Klekota-Roth fingerprint. Graph invariants/topological indices descriptors comprise preferably at least one of topostructural indices and topochemical indices.

In a preferred embodiment the polymer physicochemical parameters are 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the polymer physicochemical parameters refer to 3D descriptors comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or non-polar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of polymer, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution. The method further comprises providing a digital representation of application conditions for the target polymer, wherein a digital representation of the application conditions is indicative or associated with application parameters defining the application conditions. In particular, the providing can refer to receiving the application conditions from an input of a user using, for instance, a respective input unit. Moreover, the providing can also refer to accessing a storage unit on which the application conditions are already stored. Furthermore, the providing can also refer to a presetting of application conditions. For example, if the method is utilized in a very specific context that is only sensible with one specific application condition, the respective application condition can be preset and thus has not to be provided as specific input. Further, the providing can also comprise receiving directly the application parameter values of the application parameter, for instance, via a network connection, from other sources and providing the received application parameter values of application parameters as application conditions.

Generally, application conditions can refer to conditions that can be present during a lifetime of the polymer, for example, during a production of a product comprising the polymer, during an intended application of a product comprising the polymer, during a waste treatment of a product comprising the polymer, etc. The application conditions can be defined by respective application parameters characterizing the application conditions. For example, the application parameters can refer to a salt concentration of a watery solution, if the application condition indicates such an environment might be present during the lifetime of the polymer, or can refer to a humidity of air, e.g., for an application of an adhesive comprising a polymer. Again, being indicative of or associated with application parameters of application conditions is defined as allowing to access the information of the application parameters. For example, the digital representation can directly comprise the application parameters, for example, in form of values for respective quantities. However, the digital representation can also be a link to the respective application parameters via which the application parameters can be accessed, or the digital representation can refer to an identifier that is associated with the application parameters and allows to utilize a respective look up storage in order to access the application parameters. Moreover, the digital representation can also refer to information that allows to derive the application parameters using one or more known relations. For example, a geolocation of an environment can be utilized as digital representation allowing to derive, using knowledge on a respective geolocation, to derive respective application parameters. In a preferred embodiment the digital representation is provided by presenting a user with a respective input possibility of an expected application environment, for example, using a drop down menu or other presentation that allows the user to select or otherwise indicate an expected application environment. The respective provided application environment can then be regarded as digital representation of application conditions from which respective application conditions can be derived, for instance, via a look up on a storage on which associated application parameters are already stored or derive based on respective known relations.

In a preferred embodiment, if the application performance measure refers to a biodegradation, the application conditions consist of a biodegradation habitat indicative of habitat descriptor values of habitat descriptors as application parameters, wherein the habitat descriptors influence a biodegradation of a polymer in the respective habitat. For example, the biodegradation habitat can refer to a general habitat, for instance, can refer to a wastewater habitat, wherein respective habitat descriptor values for the habitat descriptors for this habitat are then already stored on a respective storage which can be accessed. However, the provided biodegradation habitat can also directly comprise the respective habitat descriptor values for the biodegradation habitat to provide a further specification of the biodegradation habitat, for example marine benthic. In this case the habitat descriptors as application parameters are indicative of environmental characteristics of the habitat. In particular, the environmental characteristics of a biodegradation habitat can influence a biological activity in the respective habitat, for example, can influence a presence, growth or absence of specific microorganisms. Thus, the environmental characteristics defined by the habitat descriptors indirectly also influence the biodegradation of a polymer in the respective habitat. For example, if a polymer is biodegradable by a specific microorganism that needs a specific salt concentration, the polymer will biodegrade fast in a habitat providing such a salt concentration, like a marine habitat, but will biodegrade much slower in a habitat without the right salt concentration, like waste water. Preferably, the biodegradation habitat refers to any one of a marine habitat, a waste water habitat, a limnic habitat, an anaerobic habitat, a compost habitat or a soil habitat. In a preferred embodiment, the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and a microbial community. In a further preferred embodiment, the biodegradation habitat refers to a limnic habitat and wherein the habitat descriptors refer to at least one of a salt concentration, a sedimentation type, oxygen level, location, sample depth a water temperature, a nutrient concentration, a pH value, an environmental type and a microbial community. In a further preferred embodiment, the biodegradation habitat refers to waste water and the habitat descriptors refer to at least one of a water temperature, a microbial community, a sludge concentration, a nutrient concentration, a pH value, a test duration and an enzyme environment. In a further preferred embodiment, the biodegradation habitat refers to soil and the habitat descriptors refer to at least one of a temperature, a sand content, a pH value, a moisture content, a nutrient concentration, a microbial community and an enzyme environment. In a further preferred embodiment, the biodegradation habitat refers to compost and the habitat descriptors refer to at least one of a temperature, compost activity, a pH value, a moisture content, humidity, compost maturity, compost composition, compost origin, a nutrient concentration, a microbial community and an enzyme environment. Generally, the habitat can also refer to a habitat of a standard test utilized for determining biodegradability of a polymer. For example, standard tests as defined by ISO13432, ISO14852, ISO14855, ISO17556 and OECD 301 also define a specific habitat in which the biodegradation takes place. Thus the providing of the biodegradation habitat can also comprise providing, for instance, selecting via a user input, one of the standard tests, wherein the habitat descriptors then refer to the specific characteristics of the test, i.e. of the test environment and thus test habitat. Moreover, the habitat can also be defined by the biodegradation of a reference polymer or other reference chemical. In this case the habitat can be provided by providing the reference and its biodegradation. In this case the reference and its biodegradation are indicative of the habitat descriptors.

In another preferred embodiment, the application performance measure is a burning behaviour. In this case the application conditions preferably refer to a standardised testing procedure for burning behaviour. For example, burning test conditions can be provided as application conditions such that it allows to derive a burning test method and burning test parameters as application parameters. For example, the burning test conditions can be provided in a form of an identifier of a burning test method, wherein based on the identifier a storage unit can be accessed comprising the burning test method and the burning test parameters. The identifier can, for example, be a name or identification number of the burning test as provided by a standard. However, the burning test conditions can also be directly provided, for instance, by a user utilizing an input unit, such that the burning test method and the burning test parameters are provided. For example, a user can be provided with a selection of known burning test methods and can then select the respective burning test method. Generally, the burning test method is indicative of a standardized test method for determining experimentally one or more burning properties of a polymer. Generally, a plurality of such standardized test methods for one or more burning properties are known and can be referred to by a respective identifier. Preferably, the burning test method refers to one of single burning item, single-flame source test, UL94, UL 94 HB, ASTM E84 and cone calorimeter test. The test parameters are further indicative of a specific test procedure of the burning test method, for instance, of one or more process parameters specifying the burning test method. For example, the test parameters can indicate the value of a distance of a heat source specified by the burning test method from the polymer. Further examples refer, for instance, to a time for which the product formed by the polymer is subjected to a heat source specified by the burning test method, a temperature profile of a heat source utilized in the burning test method, the presence or absence and specification of material between the heat source specified by the burning test method and the product formed from the polymer, specifics of a cooling source present during the burning test method, etc. In particular, the test parameters are specific to each burning test method and thus depend on the definition of the burning test method. However, even in a standardized test procedure the test parameters can be varied in some cases such that it is possible that for the same burning test method different test parameters can be specified within a range of possible test parameters defined by the burning test method.

In a further preferred embodiment, the application performance measure is an adhesion property. In this case the application conditions refer preferably to application conditions. In particular, application parameters can be defined that define the application conditions or procedure of applying an adhesive. Preferably, the application parameters quantify at least one of a humidity, one or more materials to be connected via the adhesive, a temperature, a contact pressure, an application duration, and a surface structure and/or texture.

In a further preferred embodiment, the application performance measure is a solubility and the application conditions refer to fluid environment. In particular the application parameters can defined the characteristics of the fluid environment. For example, the application parameters can quantify at least one of, a kind of fluid, a concentration of an ingredient, like salt, a temperature and a pressure.

In a further preferred embodiment, the application performance measure is a formulation property and the application conditions refer to the formulation. In particular the application parameters can defined the characteristics of the chemical composition of the formulation. For example, the application parameters can quantify at least one of, a kind of solvent, a concentration of an ingredient, like salt or co-solvent, a temperature and a pressure.

In a further preferred embodiment, the application performance measure is a washing performance measure and the application conditions refer to the washing environment. In particular, the application parameters can define, a hardness of water, a substance to be removes, a substance and/or structure of the object to be washed, washing additives, a washing process utilized, a washing temperature and a washing pressure.

The method further comprises providing a determination model based on the provided application conditions. In particular, it is preferred that the providing of the determination model refers to a selecting of a determination model based on the provided application conditions. For example, a plurality of determination models can be stored on a storage, wherein each determination model has been trained for one application condition, in particular, for different values or value ranges of application parameters. Based on the provided application conditions indicative of the application parameters, a respective suitable determination model can then be selected from the plurality of determination models. For example, a determination model is suitable if the indicated application parameters fall within the ranges of the application parameters for which the determination model has been trained. For example, a respective lookup table can be provided that allows for an easy comparison between the indicated application parameter values and/or value ranges for which the determination models stored on the storage have been trained such that directly a suitable determination model can be selected. However, in another embodiment the providing of a determination model based on the provided application conditions can also refer to a user selection of the determination model. For instance, the user can be provided with a preselection of determination models that refer to the provided application conditions and then be allowed to select the respective determination model that should be utilized. Generally, the possible stored determination models refer to determination models that have already been parameterized based on a respective training data set for one or more application conditions. Since the training data sets utilized for parameterizing a determination model are historical data, as described in more detail below, the determination models can be trained and thus generated at any time before the determination of a specific application performance measure for a specific polymer, and after the training be stored on a respective database. However, the training and thus the generation of a determination model can of course also be performed at the time that it is determined that a specific determination model, for instance, for a specific application, is needed.

The provided determination model is then adapted to determine an application performance measure of a polymer with respect to the application conditions. In particular, the determination model is a data driven model that is parameterized with respect to the application conditions such that it can determine the application performance measure of a polymer based on the digital representation of the polymer, preferably, based on the polymer characterizing parameters, for example, quantifying the physicochemical characteristics of the polymer indicated by the digital representation. The term “such that” is to be interpreted here such that the parameterization adapts and thus enables the determination model to provide the application performance measure with respect to application conditions when provided with, for example, polymer characterizing parameters as input. For example, the determination model relates polymer characterizing parameters of historic digital representations of synthesis specifications and historic digital representations of application conditions to a respective application performance measure. This allows that, based on a target application performance measure, a digital representation of the synthesis specification may be determined. The term “data driven” is used here to emphasize that the model is mainly based on respective data input and not, for instance, on intuition, personal experience or knowledge. Preferably, the determination model refers to a machine learning based model that is based on known machine learning algorithms, like neural networks, regression models, classification algorithms, etc. It has been found that for most applications in this context, in particular, regression models based on Linear Regression, Random Forests, Boosted Trees, Lasso, Ridge Regression and MARS algorithms are suitable, whereas for classification models, in particular, Random Forests, Logistic Regression and SVM algorithms are suitable. Generally, the determination model is parameterized during a training process in which, for example, characterizing parameters, preferably, polymer descriptors derived from parameters quantifying the physicochemical characteristics of the polymer, are utilized together with corresponding application performance measure for specific application conditions. Based on such a training data set that is specific for an application condition, for instance, for specific application parameter value ranges and/or values, the respective parameters of the data driven model can be determined utilizing known training methods such that the determination model is also able to determine an application performance measure of polymers that are not part of the training data set.

Moreover, in a preferred embodiment, the determination model can also be adapted to determine the application performance measure for a polymer further based on application parameters as input. In particular, the determination model can be trained by utilizing a training data set comprising, for instance, polymer characterizing parameters of polymers and associated application performance measures for a specific application condition, as described above, leading to a determination model that indirectly takes the specific application conditions into account. However, the training data set can optionally also comprise specific application parameter values of a respective application condition. In this case, the determination model can be trained such that in addition to the polymer characterizing parameters also application parameter values can be provided as input, wherein the determination model then determines the application performance measure further based on the application parameter values. This has the advantage that the application performance measure can be determined even more accurately, in particular, in cases in which the application performance measure strongly depends on the application parameter values of the application condition. For example, a biodegradability in a marine habitat can strongly deviate for different regions of the world with different temperature or nutrient concentration. Thus, for such cases it can be advantageous to directly provide the application parameter values as input to the determination model. However, it is also possible instead of providing the application parameter values as input to the determination model, to train two different determination models and indirectly treat the different regions as different application conditions.

Further, the method comprises determining the application performance measure of the polymer based on the provided determination model and the digital representation of the polymer. In particular, if the digital representation of the polymer comprises the polymer characterizing parameters, the polymer characterizing parameters are provided as input to the determination model, wherein the determination model then provides the determined application performance measure as output. If the digital representation does not directly comprise the polymer characterizing parameters, the determining of the application performance measure can comprise also determining firstly the polymer characterizing parameters, for instance, as described above. The such determined polymer characterizing parameters can then be provided to the determination model as input. However, the determination model can also be trained to directly utilize the digital representation as input, for example, wherein the determination model can then determine the characterizing parameters as part of the processing for determining the application performance measure.

The determined application performance measure can then be provided, for instance, to an output unit or to a computing unit for further processing. Preferably, the providing of the application performance measure leads to a further processing utilizing the determined application performance measure. In such a case, the providing as individual step can be omitted and replaced by the processing of the determined application performance measure.

The determination of the application performance measure utilizing the determination model can be regarded as a virtual measurement of the application performance measure. In particular, the determination model is based on measurement data, for example, measured application performance measure of polymers utilized for the training of the determination model. Thus, the determination model comprises the information provided by these previous measurements. Moreover, the characterizing parameters can in some cases also refer to measured characteristics of the polymer. Accordingly, also the determined application performance measure of a new polymer determined utilizing the determination model can be regarded as being based at least partly on measurement results.

Preferably, the processing of the application performance measure comprises determining control signals for controlling a production process based on the determined estimated application performance measure. The production process can refer to a production process of the polymer or can refer to any other process, for example, a waste treatment process, in which the polymer is utilized or processed. In a preferred embodiment the control signal is indicative of a machine executable synthesis specification of the polymer, in particular, when a comparison indicates that the determined application performance measure of the polymer lies within a predetermined range around a provided target application performance measure.

Moreover, the process of processing the application performance measure can also refer to a step of selecting one or more polymers based on respectively determined application performance measure. For example, if for a plurality of potential polymers respective application performance measure have been determined, the selecting can comprise comparing the application performance measure of the different polymers to predetermined selection criteria and select the polymers for which the determined application performance measures fulfill these criteria. In particular, in an embodiment the method comprises receiving a target application performance measure for a polymer and comparing the received target application performance measure with a determined application performance measure and providing depending on the comparison a control signal.

The control signal can refer to any signal that allows for a further control of a technical system. For example, the control signal can be adapted to control an interface for providing the result of the comparison on the interface. In a preferred embodiment the comparison refers to a validation of the target application performance measure, wherein the validation is positive if the determined application performance measure falls within a predetermined range around the target application performance measure. In this case the control signal can be adapted to simply control a user interface to provide an indication of a positive or negative validation result. However, preferably, the control signal refers to a recipe, i.e. synthesis specification, of the one or more polymers which fulfill the specified target application performance measure, i.e. which are validated positively. A recipe, i.e. synthesis specification, is generally defined as an instruction on how a polymer can be synthesized. In particular, the recipe comprises the starting substances and the respective parameters for polymerization from the starting substances. Preferably, the control signals comprise a recipe in a form that directly allows an automatic controlling of respective industrial systems or labor equipment for producing the polymer. In particular, it is preferred that the control signal is indicative of a machine executable synthesis specification of the polymer, when the result of the comparison refers to the determined application performance measure being within a predetermined range around the target application performance measure.

In a preferred embodiment the method further comprises providing as digital representation of the polymer a synthesis specification and determining the characterizing parameters, for example, in form of polymer physicochemical parameters, preferably, as polymer descriptors, from the synthesis specification. In particular, the synthesis specification, i.e. recipe, comprises information on the polymer synthesis of the polymer, for instance, on the starting substances and process by which respective starting substances are covalently bonded to form the polymer chain or network of the polymer. The method then comprises determining characterizing parameters, for instance, the polymer descriptors, from the synthesis specification. Optionally, from a synthesis specification the subgroups can be determined and the polymer characterizing parameters can then be determined based on subgroup characterizing parameters of the subgroups, for instance, from a database or utilizing known characterizing parameter determination algorithms. In a preferred embodiment, further from the synthesis specification utilized catalysts and/or non-reactive process ingredients are determined. In this case it is preferred that this information is also utilized together with the characterizing parameters, in particular, the polymer physicochemical parameters, by the determination model for determining the application performance measure. Preferably, characterizing parameters are also determined for the catalysts and/or non-reactive process ingredients and the respective characterizing parameters are also used for determining the characterizing parameters of the polymer. Preferably, the characterizing parameter for the catalysts and/or non-reactive process ingredients refers to an amount of the respective ingredient, for example, a molar mass, a molar percentage, etc. and is taken into account for determining a polymer characterizing parameters for the polymer.

In a preferred embodiment, the determining of polymer characterizing parameters from the synthesis specification comprises identifying types and amounts of subgroups based on the synthesis specification, for instance, as characterizing parameters of the subgroups, and determining the polymer characterizing parameters based on the identified types and amounts of subgroups. Generally, the types of subgroups can refer to predetermined types or classes that are associated with specific physicochemical characteristics, i.e. physicochemical parameters, of the subgroups, and thus with specific physicochemical characteristics of a polymer comprising these subgroups. However, since the general physicochemical characteristics of a polymer and hence the polymer characterizing parameters can also depend on the amount of a subgroup present in the polymer that amount can also be taken into account. In a preferred embodiment the determination of the type and amount of subgroups takes into account information provided by the synthesis specification indicative of the type of polymerization. The information on the type of polymerization that can be utilized can refer, for instance, to whether the polymerization refers to a polycondensation, polyaddition, radical polymerization, cationic polymerization, anionic polymerization, or coordinative chain-polymerization. Preferably, for each type of polymerization rules are predetermined that can be applied to determine the subgroups of the polymer. For example, rules can be predetermined that determine which functional groups of monomers in the synthesis specification react with which prioritization to which functional groups of the synthesized polymer. The rules can be based, for instance, on kinetic considerations. Based on the number and type of polymerized functional groups the subgroups can be determined and a number and type of the subgroups can be calculated.

In an embodiment the determination of the amount of subgroups comprises determining the amount of at least one of, amide, ester, thioester, carbonate, ether, amine, urea, urethane, thiourethane, isocyanurate, biuret, allophanate, acetal, Michal-adduct, radically polymerized double bond, siloxane, silane, silazane, phosphazene groups as well as residual amine, aldehyde, ketone, epoxide, aziridine, isocyanate, alcohol, thiol, carboxylic acid, acyl halogenide, α,β-unsaturated carbonyl groups, α,β-unsaturated carboxyl and double bond groups in the polymer based on the synthesis specification.

In an embodiment, the digital representation of the application conditions refers to a representation of a predetermined norm and/or testing procedure with respect to the application performance measure and the application parameters define the predetermined norm and/or testing procedure. Generally, a plurality of standardized test methods and/or norms exists for testing respective application performance measures of a chemical. For example, such test methods can be found in DIN or ISO norms. Further providing an application performance measure test method and providing a determination model that has been trained based on the provided application performance measure test method allows to determine an application performance measure that is easily comparable, for instance, with respectively measured application performance measures utilizing the respective test method. In particular, for this embodiment it is preferred that the determination models are trained based on data sets in which the test method and/or norm based on which the application performance measure has been determined is clearly specified such that a determination model can be trained specifically for one or more test methods and/or norms. For example, the predetermined norm and/or testing procedure can be a biodegradation test method, wherein the provided biodegradation test method is indicative of a standardised biodegradation test method for determining experimentally a biodegradation of a chemical. In another embodiment the predetermined norm and/or testing procedure can be a burning test method. Preferably, the burning test method refer to at least one of, but is not limited to, single burning item (EN 13823:2015), single-flame source test (EN ISO 11925-2:2020), UL94 and UL 94 HB (EN 60695-11-10: 2014 and EN 60695-11-20:2016, respectively), an ASTM E84 test, as well as cone calorimeter (ISO 5660-1:2015).

In an embodiment, further a target application of the polymer is provided referring to an intended application of the polymer, wherein the application conditions are provided based of the target application. A target application of a polymer can refer, for instance, to an intended application context of the polymer, for example, if it is intended to utilize the polymer as a coating, in personal care products, in a washing detergent, in a lubricant, in agriculture or in a packaging of a product. Such target applications indicate specific application conditions. For example, for a packaging of a product it could be interesting if a polymer biodegrades in a compost leading as application condition to a respective habitat for determining the biodegradation as application performance measure. In this context, a predetermined list can be provided on a storage on which respective target applications and corresponding application conditions and associated application performance measures are stored. A target application for a polymer can then be provided, for instance, by providing the list of target applications to a user and allowing the user to select a respective target application, wherein a respective target application is connected to one or more application conditions. An application performance measure can then be determined for each of the application conditions to which the target application is connected or again a user can select a respective application condition connected with the target application. Additionally or alternatively, information indicative of an intended treatment of the polymer can be provided. For example, an end-of-life treatment can be indicative of, whether the polymer is intended to biodegrade in a specific environment, or should be subjected to a specific treatment, for example, in a bioreactor. Thus, also the information of an intended treatment can be utilized to determine an application condition for the polymer, as described above.

In an embodiment, further information indicative of an accessible surface area of the polymer in its intended form is provided, wherein the determination model is further trained to determine an application performance measure based on the accessible surface area, and wherein the method further comprises determining the application performance measure further on the accessible surface area. For example, the information can refer to whether the intended product is provided in a solid, pulverized, foamy, pelletized, or any other form. Preferably, the information is indicative of a surface area of the product per mass or a geometry of a smallest independent part of the product. Generally, although the application performance measure of a polymer is an intrinsic characteristic of the polymer, the exact specific of an application performance measure of a product comprising the polymer can also depend on the surface area that can be accessed, for instance, by microbial components for biodegradation or for solving the polymer. Thus, further determining the application performance measure based on a surface area of a product comprising the polymer allows to increase the accuracy in the prediction of the application performance measure of the final product.

In an embodiment, polymer characterizing parameters indicated by the digital representation of the polymer refer at least to one of recipe parameters from polymer synthesis, constitutional descriptors, count descriptors, list of structural fragments, fingerprints, graph invariance, 3D-descriptors and/or higher dimensional descriptors that are indicative of a chemical nature of the polymer. Respective connections of the digital representation with polymer descriptors, for instance, calculated previously, or further information on the polymer, can be stored already and connected with the respective digital representation. For example, if the digital representation refers to a brand name, a respective structural formula, subgroups and/or subgroup characterizing parameters or polymer characterizing parameters corresponding to the brand name can be stored already, for example, on a storage of the brand name owner.

In an embodiment, the application conditions refer to any one of a biodegradation habitat, as standardised testing procedure for burning behaviour, a product application environment, and a fluid environment, and the application performance measure refers respectively to any of a biodegradability, a burning behaviour, an adhesion property and a solubility. Respective examples for this embodiment are provided above in the context of the already described embodiments.

In a further aspect, a computer implemented method is presented for determining a target synthesis specification indicative of a target polymer comprising a target measure for an application performance, wherein the method comprises a) providing a target measure for a target application performance of a polymer, b) providing a digital representation of a potential target synthesis specification indicative or associated with a potential target polymer, c) providing a digital representation of application conditions, wherein the digital representation is indicative of application parameters defining the application conditions, d) providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, e) determining the application performance measure of the potential target polymer based on the provided determination model and the digital representation of the potential target synthesis specification, and f) comparing the determined application performance measure of the potential target polymer with the target measure and, based on the comparison, either i) determining the potential target polymer as the target polymer and the potential target synthesis specification as the target Synthesis specification, or ii) providing a new potential target synthesis specification of a new potential target polymer and repeating the determination of the application performance measure utilizing the new potential target synthesis specification of the new potential target polymer.

Generally, also this method refers to a computer implemented method and can thus be performed by a general or dedicated computer adapted to perform the method, for instance, by executing a respective computer program. The method is adapted to determine, in particular, predict, a target synthesis specification indicative of a target polymer comprising a target measure. Generally, a synthesis specification includes instructions on how a specific associated polymer can be produced. For example, a synthesis specification can refer to starting products and production conditions that, if applied, lead to a synthesis of the polymer in the production process. Thus, a synthesis specification is associated always with the polymer that is produced when performing the synthesis specification, for instance, utilizing suitable laboratory or industrial equipment. In particular, a synthesis specification can also be regarded as a recipe for how to produce the associated polymer. Since the target synthesis specification and the target polymer correspond to each other, i.e. the target synthesis specification, when executed accordingly, produces the target polymer, in the following both terms can be utilized concurrently, for example, when a target synthesis specification is determined the target polymer is also determined and vice versa.

Generally, the method can be regarded as utilizing the method of determining an application performance measure as described above for steps b) to e) and providing additional steps for the providing and comparing of the target measure. In particular, the same determination model can be utilized for determining the application performance measure. Thus, all definitions provided with respect to the method for determining an application performance measure can also be applied to this method.

In a first step the method comprises providing a target measure that is indicative of an application performance measure of a polymer. In particular, the providing can refer to receiving the target measure from an input of a user applying, for instance, a respective input unit. Moreover, the providing can also refer to accessing a storage unit on which a target measure is already stored. Further, the providing can also comprise receiving a target measure, for instance, via a network connection from other sources and providing the received application performance measure. Generally, the target measure can refer to one target value, for instance, a target half-life of a polymer in a specific biodegradation habitat, or can refer to a value range that should be met by the polymer. Moreover, the target measure can also refer to any kind of target function, for instance, a timely sequence of application performance measures. For example, the target measure can indicate that the target polymer shall have a first application performance measure value range during a first time range and then a second target application performance measure value range during a following time range.

The potential target synthesis specification can be provides as described above with respect to the method for determining an application performance measure utilizing a digital representation. Generally the potential target synthesis specification can be regarded as a starting synthesis specification that indicates for which polymer or in which region of a potential polymer space the application performance measure should be determined first in the search for a synthesis specification that leads to a polymer that comprises the target measure. Generally, the potential target synthesis specification can be provided by a user or automatically, for example, in accordance with predetermined rules or also arbitrarily. For example, the user can select a promising potential target synthesis specification as starting point. However, also an arbitrary target synthesis specification can be utilized or a set of rules can be utilized for providing a potential target synthesis specification without user intervention. Preferably, the potential target synthesis specification is provided based on rules taking constrains on a potential target synthesis specification space, i.e. target polymer space, into account. The target polymer space can be defined by amount ranges of ingredients or ingredient combinations or ratios. Further, the target polymer space can be defined by process condition ranges or process condition combinations or ratios. Further, the target polymer space could be defined by synthesizability measures, which could be rule or experience based or provided by a data-driven model.

After determining the application performance measure, the determined application performance measure of the potential target polymer is compared with the target measure. Based on the comparison it is decided if the potential target polymer is determined as the target polymer and the potential target synthesis specification is determined as the target synthesis specification, wherein in this case the iteration can stop at this point. Moreover, based on the comparison it can also be determined to provide a new potential target synthesis specification of a new potential target polymer and to repeat the determination of the application performance measure utilizing the new potential target synthesis specification of the new potential target polymer. Thus, at this point an iteration is performed in which the determination of the application performance measure using the determination model and the polymer characterizing parameters of potential target polymers is repeated until one of the potential target polymers is determined as the target polymer. In particular, the comparison can comprise determining whether the determined application performance measure of a potential target polymer lies within a predetermined range around the target measure, wherein in this case the target can be regarded as being fulfilled and the potential target polymer is determined as target polymer. If the determined application performance measure lies outside of the predetermined range around the target measure, it is determined that the target is not fulfilled and a new potential target synthesis specification of a new potential target polymer is provided that might fulfil the target measure.

Generally, the performed iteration can refer to an arbitrary search of the potential target polymer space or to a directed search. For example, a new potential target synthesis specification or a new potential target polymer can simply be selected arbitrarily from a huge amount of in-silico generated potential target polymers. However, also specific rules for generating a new potential target polymer and thus a new potential target synthesis specification can be applied based on the comparison between the determined application performance measure and the target measure, with or without considering the simultaneous optimization of additional target properties of the polymer. Generally, known methods for generating new target polymers can be utilized, for example, evolutional algorithms or Bayesian optimizers can be used.

The iteration can then be performed over the steps of determining the application performance measure of the new potential target polymer by utilizing the application conditions and the polymer characterizing parameters of the new potential target polymer as descript above. Optionally, also a determination of polymer characterizing parameters from the digital description of the new potential target synthesis specification can be part of the iteration, if the polymer characterizing parameters are not already provided with the digital description of the new potential target synthesis specification. Moreover, it is preferred that the same determination model is used in all iteration steps for determining the application performance measure. However, in some cases also different determination models can be used in different iteration steps. For example, if other polymer physicochemical parameters for the new potential target polymer are utilized also another determination model can be more suitable.

After the iteration has stopped, for instance, after the potential target polymer has been determined as the target polymer, or if no new potential target polymer can be selected or generated, the result of the iteration can be provided to a user. For example, if none of the possible potential target polymers has met the target measure, the user can be notified of the failure of determining a target polymer. In case a target polymer can be determined, the target polymer can be provided to the user as output. For example, the determined target polymer and target synthesis specification can then be provided to an output unit or to a computing unit for further processing. Preferably, the providing of the target synthesis specification and the target polymer leads to a further processing utilizing the target synthesis specification.

Preferably, the processing of the target synthesis specification comprises determining control signals for controlling a production process based on the determined target synthesis specification. Preferably, the production process refers to a production process of the target polymer utilizing the target synthesis specification. Moreover, it is preferred that the target specification refers to a machine executable synthesis specification of the target polymer such that the control signals can directly refer to a controlling of respective laboratory or process equipment allowing to execute the synthesis specification to produce the polymer. In an embodiment, the providing of the target synthesis specification of the target polymer comprises providing control signals adapted for controlling an industrial plant for producing the target polymer in accordance with the target synthesis specification.

In an embodiment, a target technical application property for the target polymer is provided and the potential target synthesis specification is provided based on the provided target technical application property such that the potential target polymer fulfils the provided target technical application property. In particular, the technical application property can refer to any property of a polymer and/or a substance consisting at least partly of the polymer, that allows to assess a technical applicability of the respective polymer as provided after its synthesis. Preferably, the technical application property comprises at least one of mechanical properties, optical properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. An optical property can generally comprise any of coloration, turbidity, opaqueness, lucidity, reflection, appearance, absorption, scattering, color strength, cloud point, matting degree, optical density, spectra, refractive index. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, and charge density. The chemical property can comprise any of functional group count, atom type count, functional group density, atom type density, chemical resistance, reaction timing, demolding time, growing, hard/soft segment content, crystallinity, reaction temperature, reaction pressure, decomposition, thermal decomposition, photodegradation, acidity, pKa, pH, moisture/water content, flammability, burning rate, self-ignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, toxicity, biotransformation, ecotoxicology, sensitization, bacterial count, enzyme activity, distribution in environment, bioaccumulation, biological expo-sure. In a preferred embodiment, the technical application property can further refer to a biodegradability, for instance, to a biodegradability in a specific habitat.

The potential target synthesis specification is then provided such that the associated potential target polymer fulfils the provided target technical application property. For example, a database can be utilized on which polymers and corresponding technical application properties are already stored and from the database target polymers and associated synthesis specifications can be selected that fulfil the provided target technical application property. Generally, the polymers fulfilling the target technical application property can be regarded as forming the potential target polymer space that can be explored during the iteration process for finding the target polymer. From the selected target polymers fulfilling the target technical application property the first potential target polymer and thus the first potential target synthesis specification can then be selected.

In an embodiment, the providing of a new potential target synthesis specification is based on amending the provided target application property and providing the new potential target synthesis specification such that the potential target polymer fulfils the amended target application property. In particular, if the new potential target synthesis specification has to be provided, the comparison of the determined application performance measure and the target measure indicates that the determined application performance measure of the current potential target polymer does not fulfil the target measure. In such a case, the new potential target synthesis specification and thus a new potential target polymer can be provided such that the new potential target polymer still fulfils the target technical application property, if such a respective polymer exists. However, in many cases it will not be possible to provide such a new potential target polymer or it might not be technically sensible to provide such a new potential target polymer that still fulfils the target technical application property. In these cases it is advantageous to amend the target application property, for instance, to utilize a less strict target technical application property, like amending the target technical application property such that it now refers instead of one specific value to a value range or if it refers to a value range to a wider value range. The new potential target polymer can then be selected or generated such that it fulfils the amended target application property.

In an embodiment, the providing of the potential target synthesis specification based on the provided target technical application property comprises utilizing an application property model adapted to determine a technical application property of a polymer based on the digital representation of the polymer, wherein the application property model is a data driven model parameterized such that it determines based on the digital representation comprising the polymer characterizing parameters of the polymer the technical application property associated with the polymer. The application property model can refer to any known data driven model that allows to determine the technical application property based on a digital representation of a polymer comprising polymer characterising parameters. Generally, it is preferred that the application property model follows the same principles as described above with respect to the determination model. In fact, the application property model can be based on or utilize the same machine learning algorithms and training methods, only utilizing different training data, i.e. training data comprising instead of the application performance measure another respective technical application property of a polymer. Thus, all embodiments described above with respect to the determination model can also be realized with respect to the application property model for determining the technical application property. Utilizing such a model has the advantage that an iteration can be performed not only over the application performance measure of a polymer but also over one or more further technical application properties in a fast and computationally inexpensive manner leading to a target polymer that not only fulfils a target measure but also the one or more further target technical application properties.

In a further aspect, an interface method for providing an interface is presented, wherein the interface method comprises i) receiving as input a digital representation and an application condition via a user interface and providing the received digital representation and the application condition to a processor performing the methods as described above, and ii) providing the determined application performance measure of the polymer or the determines synthesis specification, respectively, to a user via a user interface as result, wherein the result is received from the processor performing the methods as described above.

In a further aspect, a computer implemented training method for training a data driven based determination model for parameterizing the determination model is presented, wherein the training method comprises i) providing training data associated with a predetermined application condition, wherein the training data comprises a) digital representations of a plurality of training polymers indicative of characterizing parameters for each of the training polymers, and b) an application performance measure for the respective application condition associated with each training polymer, ii) providing a data driven based trainable determination model, iii) training the provided data driven based determination model based on the provided training data such that the trained determination model is adapted to determine an application performance measure of a polymer based on a digital representation of the polymer, in particular, based on the characterizing parameters, preferably, polymer physicochemical parameters, more preferably, polymer descriptors, indicated by the digital representation, and iv) providing the trained determination model.

In a further aspect, an apparatus is presented for determining a measure for an application performance of a target polymer, wherein the apparatus comprises a) a digital representation providing unit for providing a digital representation of a target polymer indicative or associated with characterising parameters of a polymer, b) an application conditions providing unit for providing a digital representation of application conditions for the target polymer, wherein a digital representation of the application conditions is indicative or associated with application parameters defining the application conditions, c) a model providing unit for providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, and d) a determining unit for determining the application performance measure of a target polymer based on the provided determination model and the digital representation of the target polymer.

In a further aspect, an apparatus is presented for determining a target synthesis specification indicative of a target polymer comprising a target measure for an application performance, wherein the apparatus comprises a) a target providing unit for providing a target measure for a target application performance of a polymer, b) a digital representation providing unit for providing a digital representation of a potential target synthesis specification indicative or associated with a potential target polymer, c) an application conditions providing unit providing a digital representation of application conditions, wherein the digital representation is indicative of application parameters defining the application conditions, d) a model providing unit for providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, e) a determining unit for determining the application performance measure of the potential target polymer based on the provided determination model and the digital representation of the potential target synthesis specification, and f) a comparing unit for comparing the determined application performance measure of the potential target polymer with the target measure and, based on the comparison, either i) determining the potential target polymer as the target polymer and the potential target synthesis specification as the target synthesis specification, or ii) providing a new potential target synthesis specification of a new potential target polymer and repeating the determination of the application performance measure utilizing the new potential target synthesis specification of the new potential target polymer.

In a further aspect, an interface apparatus for providing an interface is presented, wherein the interface apparatus comprises i) an input interface unit for receiving as input a digital representation and application conditions via a user interface and for providing the received digital representation and the application conditions to at least one of the apparatuses as described above, and ii) a result interface for providing the determined application performance measure of the polymer to a user via a user interface as result, wherein the result is received from the apparatuses as described above.

In a further aspect, a training apparatus for training a data driven based determination model for parameterizing the determination model is presented, wherein the training apparatus comprises i) a training data providing unit for providing training data associated with a predetermined application condition, wherein the training data comprises a) digital representations of a plurality of training polymers indicative of characterizing parameters for each of the training polymers, and b) an application performance measure for the respective application condition associated with each training polymer, ii) a trainable model providing unit for providing a data driven based trainable determination model, iii) a training unit for training the provided data driven based determination model based on the provided training data such that the trained determination model is adapted to determine an application performance measure of a polymer based on a digital representation of the polymer, in particular, based on the characterizing parameters, preferably, the polymer physicochemical parameters indicated by the digital representation, and iii) a trained model providing unit for providing the trained determination model.

In a further aspect of the invention a use of at least one of the above described methods and apparatuses as described above is presented, wherein the methods and/or apparatuses, respectively, are used for determining an application performance measure for a predetermined polymer or for determining target synthesis specifications for any of the following i) polymers referring to polyesters, in particular, used for mulch film and packaging applications, e.g. aromatic aliphatic copolyesters, ii) polymers referring to polyalkoxylates, in particular, used for home and personal care applications, iii) polymers referring to polyurethane dispersions, iv) polymers used for aroma applications, v) polymers used for paper coatings for packaging applications based on multilayer blends, and vi) polymers referring to polyurethane used for adhesives.

In a further aspect of the present invention, a system is presented, wherein the system comprises i) a control signal comprising a synthesis specification of a polymer indicating one or more ingredients for producing the polymer, wherein the control signals are generated according to any of the above described methods, and ii) the one or more ingredients indicated by the synthesis specification in the control signal.

In a further aspect of the invention, a use of a control signal generated according to the above described method for controlling a production process, in particular, a production process comprising the production of a polymer is presented.

In a further aspect of the invention, a control signal is presented, wherein the control signal is generated according to the above described method. Preferably, the control signal comprises a machine executable synthesis specification for producing a target polymer.

In a further aspect, a computer program product for determining an application performance measure for a predetermined polymer is presented, wherein the computer program product comprises program code means for causing the apparatus as described above to execute the method as described above.

In a further aspect, a computer program product for determining a target synthesis specification is presented, wherein the computer program product comprises program code means for causing the apparatus as described above to execute the method as described above.

In a further aspect, a computer program product for training a determination model is presented, wherein the computer program product comprises program code means for causing the training apparatus as described above to execute the training method as described above.

It shall be understood that the methods as described above, the apparatuses as described above and the computer program products as described above have similar and/or identical preferred embodiments, in particular, as defined in the dependent claims. Moreover, also the training method as described above, the training apparatus as described above and the training computer program product as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system comprising an apparatus for determining a target synthesis specification indicative of a target polymer comprising a target application performance measure,

FIG. 2 shows schematically and exemplarily a flow chart of a method for determining a target synthesis specification indicative of a target polymer comprising a target application performance measure,

FIG. 3 shows schematically and exemplarily a flow chart of a method for training a determination model for determining an application performance measure of a polymer,

FIGS. 4 and 5 show schematically and exemplarily a flow chart of preferred more detailed embodiments of a method for determining a target synthesis specification indicative of a target polymer comprising a target measure,

FIG. 6 shows schematically and exemplarily an optional extension of a method for determining an application performance measure of a polymer,

FIGS. 7 to 9 show schematically and exemplarily a block diagram of a system architecture of a system and apparatus for determining an application performance measure of a polymer, and

FIG. 10 shows schematically and exemplarily an output and input screen of an exemplary user interface.

### DETAILED DESCRIPTION OF EMBODIMENTS

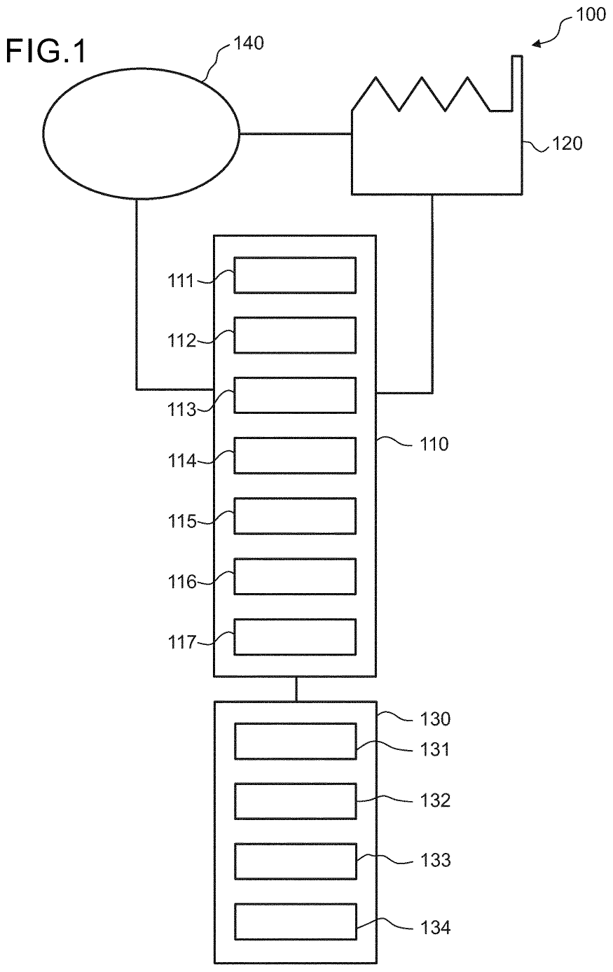

FIG. 1 shows schematically and exemplarily an embodiment of a system 100 comprising an apparatus 110 for determining a target synthesis specification indicative of a target polymer comprising a target measure, for example, a target biodegradability. Further, the system 100 comprises a training apparatus 130 for training a biodegradation model utilized in the apparatus 110, a database 140 on which results of the determining of the target synthesis specification can be stored and a production system 120 for producing a product, in particular, comprising the determined target polymer that can be controlled utilizing the determined target synthesis specification. Generally, the apparatus 110 for determining a target synthesis specification can be regarded as comprising an apparatus for determining an application performance measure comprising some of the logical units and functions of the apparatus 110.

The apparatus 110 comprises a target measure providing unit 111, a digital representation providing unit 112, an application condition providing unit 113, a model providing unit 114, a determining unit 115, an iteration control unit 116 and optionally an output and/or control unit 117 that can be adapted to output the determined target synthesis specification and/or to provide control signals for controlling a production process of the production system 120 based on the determined synthesis specification. In particular, the digital representation providing unit 112, the application condition providing unit 113, the model providing unit 114, and the determining unit 115 can be regarded as forming an apparatus for determining an application performance measure being part of the apparatus for determining a target synthesis specification indicative of a target polymer comprising a target measure.

The target measure providing unit 111 is adapted to provide a target measure indicative of an desired application performance measure of a polymer. The target measure providing unit 111 can refer, for instance, to an input unit into which a user can input a respective target measure. Moreover, the target measure providing unit 111 can refer to or can be part of a user interface that allows the user to interact with the apparatus 110 for providing the target measure. However, the target measure providing unit 111 can also refer to or be communicatively coupled with a storage unit on which a target measure, for instance, for a specific application, is already stored.

The digital representation providing unit 112 is adapted to provide a digital representation indicative of a potential target synthesis specification indicative of polymer characterizing parameters of a potential target polymer. The digital representation providing unit 112 can refer, for instance, to an input unit into which a user can input the respective digital representation. Moreover, the digital representation providing unit 112 can refer to or be part of a user interface that allows the user to interact with the apparatus 110 and/or the database 140. However, the digital representation providing unit 112 can also refer to or be communicatively coupled with a storage unit on which the digital representation of the polymer is already stored. Generally, the digital representation can directly comprise characterizing parameters, for instance, polymer physicochemical parameters that are indicative of parameters quantifying physicochemical characteristics of the respective polymer. However, instead of directly providing the polymer characterizing parameters also only the synthesis specification of a polymer can be provided. In this case, it is preferred that the digital representation providing unit 112 is further adapted to determine the polymer characterizing parameters from the synthesis specification. In particular, it is preferred that the digital representation providing unit 112 is adapted to identify from the potential target synthesis specification types and amounts of subgroups of the polymer and to determine the polymer characterizing parameters based on the identified types and amounts of subgroups. In particular, the digital representation providing unit 112 can be adapted to determine for each identified subgroup respective subgroup characterizing parameters, for instance, by accessing a database on which for a plurality of the most relevant subgroups respective characterizing parameters are stored. The characterizing parameters of the polymer can then be determined based on the subgroup characterizing parameters of the subgroups and preferably, also on the determined amount and type of the subgroups, for example, by weighted averaging of the subgroup characterizing parameters of the subgroups. The digital representation providing unit 112 is then adapted to provide the digital representation comprising the polymer characterizing parameters, for instance, to the determining unit 115.

The application condition providing unit 113 is adapted to provide application conditions. The application condition providing unit 113 can refer, for instance, to an input unit into which a user can input a respective application condition. For example, a user interface can be provided that allows a user to select from a number of predetermined application conditions. In a preferred embodiment, the application condition providing unit 113 can be communicatively coupled to or refer to a user interface that allows to indicate an application intended for the polymer, wherein the application condition providing unit can then be adapted to provide an application condition based on the intended application. For example, if the application indicates the utilization in a washing detergent the application condition can refer to a habitat, for example, waste water, in which the polymer is intended to biodegrade. The application condition providing unit can then be adapted to determine as application condition a respective waste water biodegradation habitat and the apparatus can determine a biodegradation of the polymer in this waste water.

Generally, an application condition is indicative of and/or defined by application parameter values of application parameters influencing an application performance measure of a polymer under the respective application conditions. In particular, application parameters are indicative of characteristics of the application condition, for example, for a marine habitat a salt concentration can strongly influence the biodegradation of a polymer in the marine habitat. Specific application parameter values typical for a respective application condition can be stored on a database. However, a user can also input respective specific application parameter values, for example, if it is known that the application parameter values for the respective habitat deviate from the typical application parameter values.

The model providing unit 114 is adapted to provide a determination model based on the provided application conditions. In particular, it is preferred that the model providing unit 114 is adapted to select the determination model from a plurality of determination models stored already on a database. For example, a determination model can be trained with respect to training data corresponding to one or more specific application conditions. These specific application conditions can be defined with respect to specific application parameter values or value ranges that define for which application condition the respective determination model is suitable. For example, a lookup table can be provided that allows the model providing unit to select based on the application condition, for instance, based on the application parameter values of the application condition, which of the determination models is suitable. However, the model providing unit 114 can also comprise or refer to an input unit to which the determination model can be provided, for instance, by a user selection or user input that indicates which determination model should be used.

The determination model is a data-driven model parameterized such that it can determine the application performance measure of the polymer based on the digital representation, preferably, based on the polymer characterizing parameters that can be indicative of the physicochemical characteristics associated with the polymer. Optionally, the determination model can also be trained to further utilize the provided application parameter values as input. In a preferred embodiment, the data-driven model refers to a machine learning model, for instance, utilizing regression model based algorithms or classifier model based algorithms. A regression model based algorithm can be based on any of a neural network algorithm, a Linear Regression algorithm, a LASSO algorithm, a Ridge Regression algorithm, a MARS algorithm, a Random Forest algorithm, and a Boosted Trees algorithm. A classifier based model algorithm can be based on any of a Random Forest algorithm, a Logistic Regression algorithm and a SVM algorithm. The inventors have found that for most applications, in particular, Linear Regression, Random Forest and MARS based algorithms are suitable.

The determination model can be trained, for instance, utilizing training apparatus 130. In particular, the training apparatus 130 comprises a training data providing unit 131 for providing training data for training the data-driven based determination model. The training data comprises a) digital representations indicative of polymer characterizing parameters of a plurality of training polymers, and b) application performance measures associated with each training polymer for one or more different application conditions. Optionally, the training data set can further comprise application condition values of the specific application condition for which the respective application performance measure of a polymer has been determined. Preferably, in the training data the application performance measure provided for each training polymer refers to an application performance measure that is measured in accordance with the same measurement method. However, application performance measures can also be provided for different measurement methods, wherein in this case it is preferably clearly indicated which application performance measures are associated with which measurement methods, such that the determination model can be trained to differentiate between different measurement methods. Generally, the training data can be designed to cover a predetermined condition space of a to be trained determination model, wherein the condition space is defined by the value ranges of the respective application parameters for which the determination model shall be trained. For example, the training data can be designed to cover predetermined polymer types for a predetermined application condition. Known methods for designing and optimizing training data for a predetermined condition space can be utilized such that the condition space is well covered with training data and that random outliers are avoided.

Further, the training apparatus 130 comprises a model providing unit 132 adapted to provide a data-driven based trainable determination model, for instance, a determination model comprising parameters that can be set during the training process for training the determination model. For example, a trainable determination model can already be stored on a storage unit to which the model providing unit 132 can have access for providing the same. Moreover, the training apparatus 130 comprises a training unit 133 for training the provided data-driven based determination model based on the provided training data. In particular, the training can refer to varying the parameters of the determination model based on the respective training data until the determination model is adapted to determine an application performance measure of a polymer based on a digital representation, in particular, based on polymer characterizing parameters. Generally, any know training algorithms for training data-driven, in particular, machine learning based models can be utilized. Preferably, during the training of the determination model also the characterizing parameters of the polymer that have the most influence on the application performance measure in the respective application condition are determined and the model is then trained based on these most influential characterizing parameters. For determining these most influential characterizing parameters, for example, cluster analysis or PCA analysis tools can be utilized. In particular, the characterizing parameters can be utilized to determine the application space of the training data, wherein the application space is then defined by the characterizing parameters of the polymer and the application parameters that are covered by the data. The determination of the most influential characterizing parameters and/or application parameters can then be performed as a dimension reduction of the application space. Then algorithms for optimizing the training data in the application space can be applied, for instance, to cover the application space with as few training data as possible.

The training apparatus 130 then comprises a trained model providing unit 134 that is adapted to provide the trained determination model, for instance, to a storage unit on which respectively trained determination models for different application conditions and/or different types of polymers, and/or polymer characterizing parameters are stored. However, the trained model providing unit 134 can also be adapted to directly provide the trained determination model, for instance, to the determination model providing unit 114 of apparatus 110.

In all cases, the determination model providing unit 114 is then adapted to provide a suitable trained determination model to the determining unit 115. The determining unit 115 can then utilize the determination model and the provided digital representation for determining the application performance measure. In particular, the determining unit 115 can be adapted to utilize the polymer characterizing parameters indicated by the digital representation as input to the determination model that has, as already described above, been trained to then provide as output a determination for the application performance measure for which it has been trained. The such determined application performance measure can then be provided for further processing. For example, the application performance measure can be processed by being displayed on a display or by generating control signals based on the application performance measure, for instance, for controlling a production process associated with the polymer. Preferably, the application performance measure is further processed by validating the application performance measure, in particular, with respect to an intended application. A respective synthesis specification can then be provided based on a result of the validation.

A preferred validation is performed by the iteration control unit 116. The iteration control unit 116 is adapted to control an iteration process for determining the target synthesis specification. In particular, the iteration control unit 116 is adapted to compare the determined application performance measure of the potential target polymer with the target measure. Based on this comparison, the iteration control unit 116 is then adapted to decide whether a further iteration step is necessary for determining a target synthesis specification or if the iteration has reached an end, in particular, if the potential target polymer can be set as the target polymer and thus the potential target synthesis specification as the target synthesis specification. Preferably, the comparing of the determined application performance measure of the potential target polymer and the target measure refers to determining whether the determined application performance measure lies within a predetermined range around the target measure, for instance, by determining whether a difference between the determined application performance measure and the target measure lies below a predetermined threshold. However, the comparison can also refer to a more complex mathematical function and the condition for which the potential target polymer is determined as the target polymer can refer to any condition that is based on the comparing of the determined application performance measure with the target measure. Generally, if the predetermined condition is fulfilled, for instance, if the determined application performance measure lies within the predetermined range around the target measure, the iteration control unit 116 determines that the potential target polymer is the target polymer and that the potential target synthesis specification is the target synthesis specification and ends the iteration.

If the above condition is not fulfilled, for instance, if the determined application performance measure lies not within the predetermined range around the target measure, the iteration control unit 116 is adapted to decide that a further iteration step is necessary. In this case, the iteration control unit 116 is adapted to provide a new potential target synthesis specification of a potential target polymer and to repeat the determination of the application performance measure utilizing the new potential target synthesis specification of the new potential target polymer. For example, the new potential target polymer synthesis specification can be provided on a database on which a plurality of potential target synthesis specifications are already stored and from which the iteration control unit 116 can select a new potential target synthesis specification arbitrarily or according to predetermined rules. Such rules can, for instance, be a function of the comparison of the determined application performance measure with the target measure of the potential target polymer. For example, the function can refer to the size of the difference between the determined application performance measure and the target measure, wherein the smaller the difference the more parts of the new potential target polymer are similar in the potential target polymer. In such a case, these rules can lead to the iteration control unit 116 being adapted to select new potential target polymers that are more similar to the potential target polymer if the determined application performance measure for the potential target polymer is already similar to the target measure and that are less similar if the difference between the determined application performance measure and the target measure is high. However, also completely different rules can be applied. Moreover, the iteration control unit 116 can also be adapted to generate a new potential target synthesis specification, for instance, based on the potential target synthesis specification and predetermined rules or arbitrarily. Also in this case for the rules the same principles as described above can be applied.

Moreover, the iteration control unit 116 can also be adapted to apply an abortion criterion for the iteration that indicates that for a respective target measure no suitable target synthesis specification can be found. For example, the iteration control unit 116 can be adapted to apply an abortion criterion that refers to a predetermined number of iteration steps, i.e. that refers to determine a predetermined number of new potential target synthesis specifications. However, also other abortion criteria can be utilized.

An output unit referring, for instance, to a display, can then be adapted to output the determined target synthesis specification or the target polymer, for instance, in form of a visual representation of the polymer, an identification of the polymer, a chemical formula representing the polymer, etc. Moreover, the output unit can additionally or alternatively be adapted to provide the determined target syntheses specification to a database 140 for storing the respective determined target syntheses specification in association with the respective target measure for a future usage. Optionally, the apparatus 110 can comprise the control unit 117 that is adapted to provide control signals based on the determined target synthesis specification for controlling a production process of the production system 120. In particular, it is preferred that the control signals are indicative of the machine executable synthesis specification of the target polymer which is generated based on the determined target synthesis specification for producing the target polymer fulfilling the target measure. However, the control unit 117 can also be adapted to control the production process of another product based on the determined target synthesis specification, for instance, to provide control signals indicative of a machine executable synthesis specification for another product utilizing or comprising the respective target polymer.

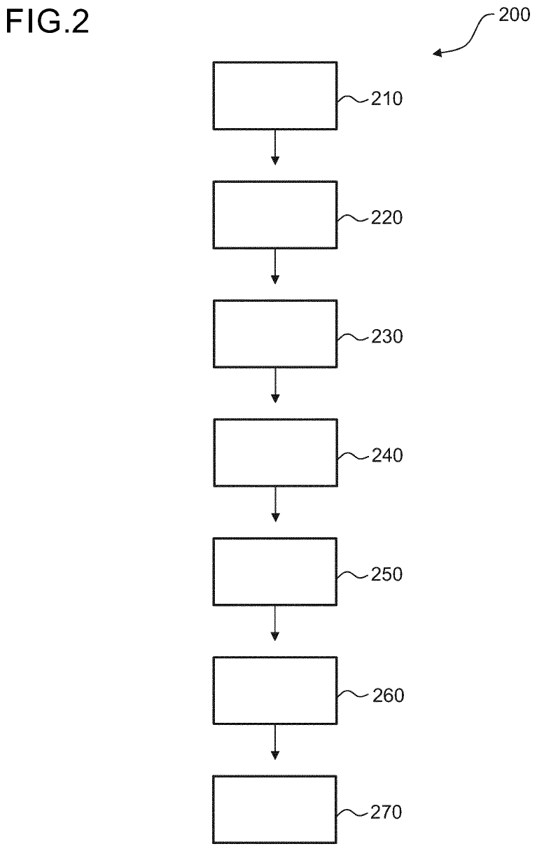

FIG. 2 shows schematically and exemplarily a flow chart of a method for determining a target synthesis specification indicative of a target polymer comprising a target measure. Generally, also this method can be regarded as comprising the steps of a method for determining an application performance measure. The method 200 comprises a first step 210 of providing a target measure. Further, in a step 220 a digital representation of a potential target synthesis specification indicative of polymer characterizing parameters of a potential target polymer are provided. In particular, the providing of the target measure and of the digital representation can be in accordance with the principles described above with respect to the target measure providing unit 111 and the digital representation providing unit 112, respectively. Further, in a step 230 an application condition indicative of application parameter values of application parameters influencing an application performance measure of a polymer in a respective application condition is provided. Also for this step 230, the principles described above, for instance, with respect to the habitat providing unit 113 can be applied. Further, in step 240 a determination model is provided that is adapted to determine the application performance measure of the polymer based on the digital representation. As already discussed above in more detail, the providing of the determination model can also refer to a selection of the determination model based on the provided application condition. Moreover, the determination model is a data driven model parameterized with respect to the application condition such that it can determine an application performance measure of a polymer based on the polymer characterizing parameters. Generally, the steps 210, 220, 230 and 240 can be performed in arbitrary order or even concurrently. In a following step 250 an application performance measure is determined based on the provided digital representation of the potential target polymer and the determination model. Steps 220, 230, 240 and 250 can be regarded as the steps of a method for determining an application performance measure, wherein after these steps the determined application performance measure can be provided for further processing, for instance, as in step 260. In a step 260 the determined application performance measure of the potential target polymer is then compared with the target measure. Based on this comparison, either the potential target polymer is determined as a target polymer and the potential target synthesis specification as a target synthesis specification, or a new potential target synthesis specification of a new potential target polymer is provided and the determination of the application performance measure utilizing the new potential target synthesis specification of the potential target polymer is repeated. In an optional step 270 after a target synthesis specification has been determined utilizing the steps above, the determined target synthesis specification together with the determined target polymer and the target measure can be provided to a user via an output unit. Moreover, in the step 270 the potential target synthesis specification can also be utilized for generating control signals that allow for a controlling of a production process of a product, for instance, of the target polymer or of a product comprising the target polymer, as already described above in detail.

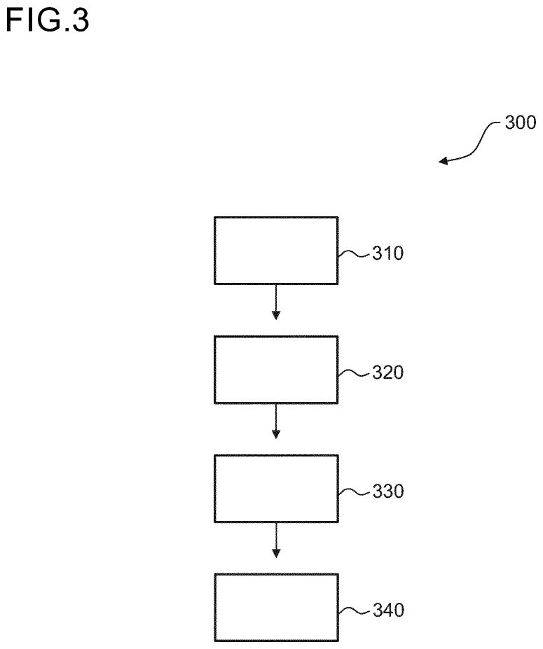

FIG. 3 shows schematically and exemplarily a flow chart of a method for training the data driven based determination model utilized, for instance, in the method 200 discussed with respect to FIG. 2. Generally, the method 300 can be performed, for instance, by respective units of the training apparatus 130 as described with respect to FIG. 1. The method 300 comprises a step 310 of providing training data for training the data driven based determination model. The training data comprises a) digital representations indicative of polymer characterizing parameters of a plurality of training polymers, and b) an application performance measure associated with each training polymer in a respective application condition, for instance, for specific application parameter values. In particular, the training data can be provided in accordance with the principles described above with respect to the training data providing unit 131 described with respect to FIG. 1. The method comprises further a step 320 of providing a data driven based trainable determination model, for instance, a machine learning based determination model like a neural network. Generally, the step 310 and the step 320 can be performed in arbitrary order or even at the same time. The method 300 then further comprises a step 330 of training the provided data driven based determination model based on the provided training data, for instance, by varying parameters in the data driven based trainable determination model, such that the trained determination model is adapted to determine an application performance measure of a polymer based on a digital representation of the polymer. In step 340 the trained determination model can then be provided, for instance, by storing the trained determination model on a storage or by directly providing the trained determination model to the apparatus 130 as described with respect to FIG. 1.

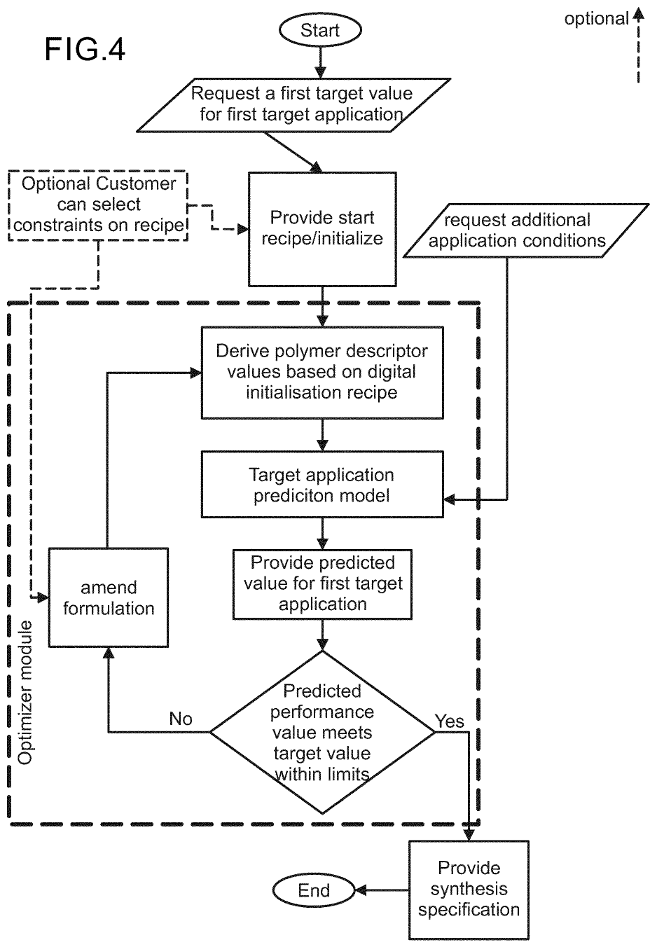

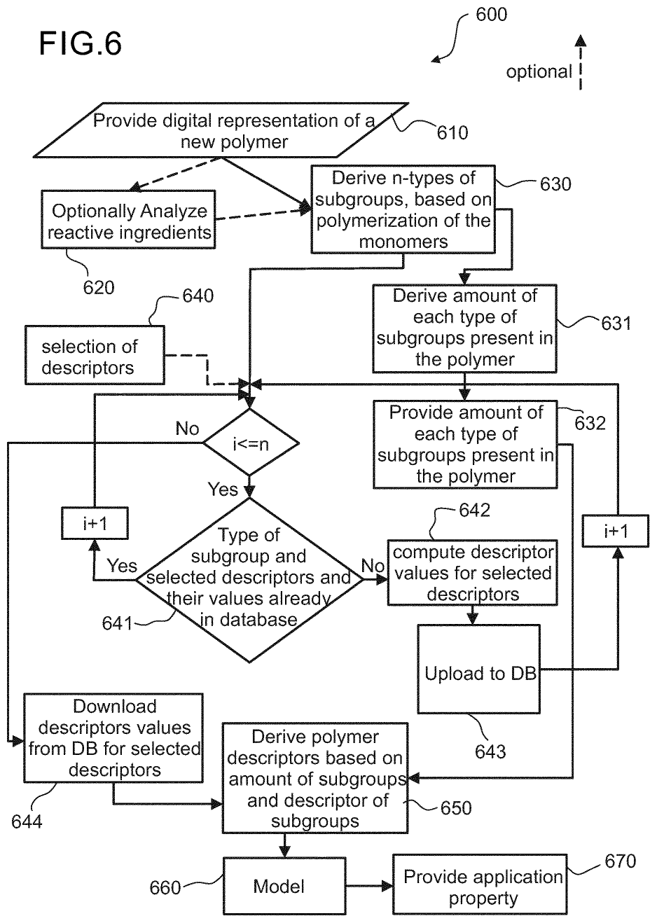

In the following, more detailed preferred examples of the above described method and the corresponding apparatus will be described. A schematic and exemplary flow chart of an exemplary and preferred embodiment of the method is provided by FIG. 4. In this exemplary embodiment, the method starts with requesting, for instance, via a user interface, a target measure for a target application, for example, a target biodegradability or a target burning property. Moreover, in a next step, the optimization is initialized by providing a potential target synthesis specification, i.e. a start recipe. Optionally, constraints on the recipe, i.e. the synthesis specification, can be taken into account in this process, for instance, if a user provides such constraints. The constraints can refer, for instance, to constraints in the production of a polymer, in the starting substances that should be used for synthesizing the polymer, etc. Moreover, application conditions are requested being, for example, indicative of a biodegradation habitat or a burning behavior test. Moreover, the application conditions can also be indicative of further information with respect to the target polymer that should be fulfilled. For example, the requested application conditions can refer to an intended application indicating the application conditions. Based on the above steps, the optimization for determining the target polymer, i.e. the target synthesis specification, can be initialized. In a first step of the optimization, polymer physicochemical parameter values can be derived from the provided start recipe, i.e. from the provided potential target synthesis specification. A more detailed, preferred possibility for deriving the physicochemical parameter values is described with respect to FIG. 6. However, the deriving of the polymer physicochemical parameters can also refer to accessing a storage on which respective physicochemical parameter values for the respective potential target polymer are already stored. Moreover, if the provided digital representation of the potential target syntheses specification already comprises the polymer physicochemical parameters, this step can also be omitted. Based on the requested application conditions a respective determination model can be provided. Based on the provided determination model and the digital representation of the potential target synthesis specification, a value for the target application, i.e. the application performance measure, for the potential target polymer can be provided. In a next step it is determined if the determined performance value, i.e. the determined application performance measure, meets the target value, i.e. the target measure, within predetermined limits. If this is not the case, i.e. if this condition is not fulfilled, the formulation of the potential target synthesis specification is amended and a new target synthesis specification is determined optionally taking into account the constraints previously provided. The iteration can then start anew for the new potential target synthesis specification. If at one point the determined performance value meets the target value within limits, i.e. if the respective condition is fulfilled, the potential target synthesis specification is determined as the target synthesis specification and provided, for example, to a user or to a control unit for producing the respective determined target polymer.

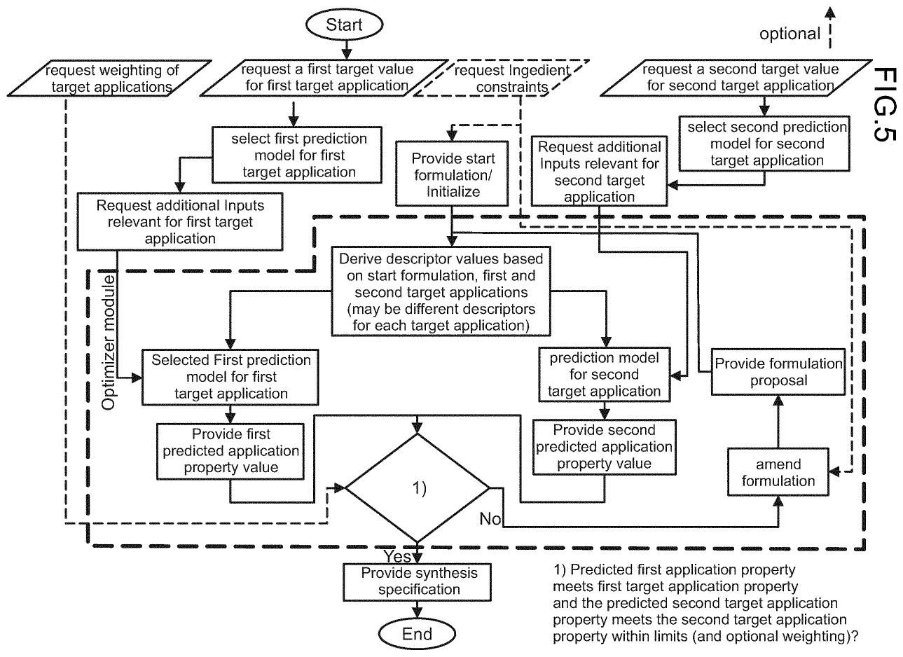

FIG. 5 shows schematically and exemplarily a further preferred embodiment of the above method for determining a target synthesis specification with predetermined target measure, wherein in this embodiment in addition to the target measure it is desired that the target polymer also fulfills a further target value, i.e. target technical application property. The additional target technical application property can refer to any technical application property, for instance, also to a biodegradability in a specific habitat, or any other technical application property. Generally, in particular, the method follows the same principles as described above with respect to FIG. 4. However, due to the additional target value, additional conditions have to be met during the optimization. Thus, in the following only the main differences with respect to the method as described above will be pointed out. In particular, in this preferred embodiment, the optimizer module does not only optimize over the first target value, i.e. over the target measure, but also over the second target value. Preferably, also for the second target value a determination model adapted for determining a value for the technical application property based on polymer physicochemical parameters is utilized. Thus, in addition to the method as described above for the second target application a second determination model, i.e. an application property model, is provided that allows to determine an application property value based on the polymer physicochemical parameters for the second target application. The second determination model can, for instance, be based on the same algorithm as the determination model described with respect to FIG. 4, and is only trained with a different data set such that it determines another property of the polymer. The comparison then refers to not only determining whether the determined application performance measure meets the target measure within limits, but also whether the determined second application property value meets the target second application property value within limits. Predetermined rules can be utilized that determine for which cases the iteration is continued, i.e. a new formulation is provided as new potential target synthesis specification and for which conditions the potential target synthesis specification is determined as the target synthesis specification. For example, a user can predetermine weights for weighting to which extents which of the conditions has to be met. For instance, it can be more important for a user that the application performance measure is met, whereas the other target application property is not so important. In this case, either the limits within which the second target application property can be met can be set broader or the meeting of this condition can be weighted less strongly. In this context also Pareto optimization methods can be utilized to find an optimal trade of between the different targets. If at one point of the iteration it is then determined that the conditions are met and fulfil the predetermined rules the respective potential target synthesis specification can be determined as target synthesis specification and provided as output to a user or can be utilized to generate a control file for producing the respective target polymer.

FIG. 6 shows schematically and exemplarily a preferred method 500 for deriving characterizing parameters, in particular, physicochemical parameter values, in this example referring to polymer descriptors, from a digital representation of a new polymer. In a first step 510 a digital representation of the polymer is provided. The digital representation can directly comprise the polymer descriptors, wherein in this case the steps shown in FIG. 6 until step 550 can be omitted. However, in many cases the polymer descriptors first have to be determined based on the provided digital representation referring in such a case, for example, to a recipe for the synthesis of the polymer, or to a chemical representation of the polymer indicating the chemical components and bonds in the polymer. In this step 510 the digital representation can comprise any one or more of the following information: an amount of monomer components; an amount of non-monomer components, like initiators, fillers, additives; a reaction condition, like temperature, vessel, pressure, stirring rate; condition profile, e.g. temperature profile, pH value, solvents; feed profiles; type of polymerization, e.g. radical, cationic, anionic, polycondensation, polyaddition, polyether formation; post-processing, like amount of components, conditions, as well as temperature and feed profiles; type of post-processing, e.g. radical, cationic, anionic, polycondensation, polyaddition, polyether formation; chemical information on components, like mixtures, connectivity of non-polymeric pure compounds, composition of polymeric pure compounds on the basis of subgroups, connectivity of the monomers associated to the subgroups in the polymeric pure components; for block-co-polymers also information, in which block each monomer and reactive prepolymer is incorporated; for structures/layered materials and composites also information in which phase/layer each component is included. If such information is not provided by the digital representation directly, in optional step 520, the reactive components and subgroups can also be derived from the digital representation, for example, from the recipe.

If the provided information indicates the presence of a mixture, then in a following step the mixture is decomposed into its pure components and each polymer component is treated as input polymer. Moreover, the polymer composition can also be transformed into mol %, weight %, volume % or, absolute mol, if necessary.

In the next step 530 the polymerizable components can be transformed into subgroups, e.g. repeating units, and the subgroups are determined as different types. For example, polymerizable subgroups can be determined based on connectivity information of non-polymeric pure compounds by using SMARTS, for instance, via KNIME workflow. Also connectivity information of all possible subgroups can be derived from connectivity information of non-polymeric pure compounds by using reaction SMARTS, for example, also via KNIME workflow

After the subgroups and their types have been determined, in step 540 the type of descriptors that should be utilized can be provided. However, the descriptors can also be determined without first selecting the type of the subgroups. In order to decrease the computational resources for the method it is preferred that in a step 541 it is determined whether subgroup descriptors associated with a respective type of subgroup are already stored in a database, for example, if entries for subgroups with identical connectivity information already exist in the database. If this is the case the respective associated subgroup descriptor can be directly downloaded, for example, in step 544. If the determined type of subgroup is not stored on the database the subgroup descriptors that are associated with a respective type of subgroup can be determined, for example, in step 542. For example, either a 3D structure of respective types of subgroups can be derived based on connectivity information and an automatic computation of subgroup descriptors can be started using, for instance, a computer cluster, or already existing machine-learning determinations can be utilized as subgroup descriptors. Generally, it is preferred that if computations on new subgroups are necessary, in step 543, the results are stored in the database after the computations are finished. Optionally further subgroup descriptors can be provided from a topological analysis of the subgroups, a quantum chemical computation, a molecular dynamics computation, coarse-grained methods, finite-element computations and kinetic simulations. In particular, polymer reaction engineering methods can be used to derive subgroup descriptors that allow to take into account a microstructure of the polymer.

In step 531 the amount of subgroups, i.e. of each type of subgroup, is determined, for example based on the provided recipe information for the polymer and provided in step 532. For example, the amount can be determined by counting an amount of polymerizable groups per polymerizable component, optionally, including prepolymers. In this case, information on polymerizable groups can be derived from non-polymeric components and the such determined amount can be added to a count of the number of, optionally, non-polymerized, polymerizable groups of the subgroups for polymeric components based on the composition of the polymeric components to determine a resulting amount. Further, it is preferred that the amount of polymerizable groups originating from agents used for post-processing after polymerization is removed from the resulting amount.

However, although it is preferred that the polymer descriptors are derived from polymer subgroups, in other embodiments of the invention the polymer descriptors can also be derived in other ways, for instance, by directly determining the polymer descriptors from the complete polymer. Moreover, the polymer descriptors for respective polymers can also be already stored on a storage unit such that the deriving of the polymer descriptors from a digital representation of the polymer can refer to determining from the digital representation information on the polymer that allows to access the database and retrieve the corresponding polymer descriptors.

Optionally the derived amounts of subgroups can be used for a further interpretation of the polymer composition. For example, a total number of polymerized functional groups, e.g. double bonds, amine groups, alcohols groups, thiol groups, carboxylic acid groups, isocyanate groups, epoxide groups, and formed functional groups, e.g. amid groups, ester groups, thioester groups, urea groups, urethane groups, thiourethane groups, ether groups, can be determined. Also the molar weighted total number of polymerized functional groups, the mass weighted total number of polymerized functional groups, the total number of residual functional groups, e.g. double bonds, amine groups, alcohol groups, thiol, groups, carboxylic acid groups, isocyanate groups, epoxide groups, the molar weighted total number of residual functional groups, the mass weighted total number of residual functional groups, the sum of all residual functional groups, the ratio between functional groups after polymerization, the number of crosslinks in polymer, the molar fraction of crosslinks in polymer, optionally, with mass-weighting as well, the average number of atoms per subgroup, optionally, per weight as well, the average number of non-H-atoms per subgroup, optionally, per weight as well, the average number of bonds per subgroup, optionally, per weight as well, the average number of bonds between non-H-atoms per subgroup, optionally, per weight as well, the average number of rotors per subgroup, optionally, per weight as well, the average number of rotors between non-H-atoms per subgroup, optionally, per weight as well, the average number of rings per subgroup, optionally, per weight as well, the average polar surface areas per subgroup, optionally, per weight as well, the average refractivity per subgroup, optionally, per weight as well, the total number of blocks, the molar size of first block, the molar size of last block, the HLB value of polymer, optionally, with area weighted HLB value, the HLB value of block with lowest HLB value, optionally, with area weighted HLB value, the HLB value of block with largest HLB value, optionally, with area weighted HLB value, the HLB value of first block, optionally, with area weighted HLB value, the HLB value of last block, optionally, with area weighted HLB value, the mass of first block, the mass of last block, the area of block with lowest HLB value, the area of block with largest HLB value, the difference of the HLB values of the blocks, optionally, with area weighted HLB value, the hydrophilic area of the polymer, the lipophilic area of the polymer, the number of arms for ring-opening-polymerization, or the length of arms for ring-opening-polymerization can be determined.

In step 550 the determined amount and type of the subgroups and the associated subgroup descriptors can be utilized to compute the polymer descriptors. For example, the polymer descriptors can be determined by one or more of molar weighted, e.g. arithmetic, harmonic or logarithmic, averaging, mass weighted, e.g. arithmetic, harmonic or logarithmic averaging, volume weighted, e.g. arithmetic, harmonic or logarithmic, averaging, surface area weighted, e.g. arithmetic, harmonic or logarithmic, averaging of the associated descriptors of the subgroups. Moreover, the polymer descriptors can be determined by determining from the associated subgroup descriptors one or more of a molar weighted standard deviation, a mass weighted standard deviation, a volume weighted standard deviation, a surface area weighted standard deviation, a molar weighted maximum value, a mass weighted maximum value, a volume weighted maximum value, a surface area weighted maximum value, a molar weighted minimum value, a mass weighted minimum value, a volume weighted minimum value, a surface area weighted minimum value, a molar weighted sum, a mass weighted sum, a volume weighted sum, a surface area weighted sum, and a maximal difference.

In step 560 the derived or provided polymer descriptors can then be provided to the trained determination model for determining the application performance measure, for example, as described with respect to FIG. 4. The determination model can be trained based on an automated statistical preprocessing of the training data, in particular, of the training polymer descriptors, for instance, utilizing feature engineering. For example, the feature engineering can comprise determining first a plurality of different polymer descriptors for the polymer, for example, based on the subgroup descriptors of the subgroups, and to preselect from this plurality of descriptors those that are with a predetermined probability relevant for the application performance measure of a polymer in a specific application condition. Based on the relevant descriptors preferably a cluster analysis is performed to identify groups of descriptors that strongly correlate. Such groups allow to select only one of the member of the groups, i.e. only one descriptor of the group, for representing the whole group of descriptors. Thus, based on the cluster analysis the number of relevant descriptors can be reduced further. The same process can optionally be performed for the application parameter in order to determine application parameters that are most relevant for the determination of an application performance measure of polymers in a specific application condition. Based on the remaining polymer descriptors and, optionally also based on the application parameters, an application space can be determined and optimized. The application of the trained determination model, for instance, to a specific application condition or polymer descriptors, can then be determined by the space spanned by the training data that forms the application space. This space can be optimized, for example, by amending the training data to cover the application space regularly, by removing strong outliers, by adding training data in parts of the space that are not yet covered, etc. This also allows to maximize the applicability space. The determination model is then trained based on the optimized training data. The determination model can generally refer to sparse, e.g. Splines, LASSO regression, PLS, and non-sparse, e.g. ridge regression, tree methods, kernel based methods, statistical learning models for relating the polymer descriptors to the application performance measure in a specific application condition. Moreover, the determination model can further provide a reliability estimation of the determination depending on the respective used determination model. In step 570 the determined application performance measure, i.e. technical application property, can then be provided to a user, for example, via a user interface.

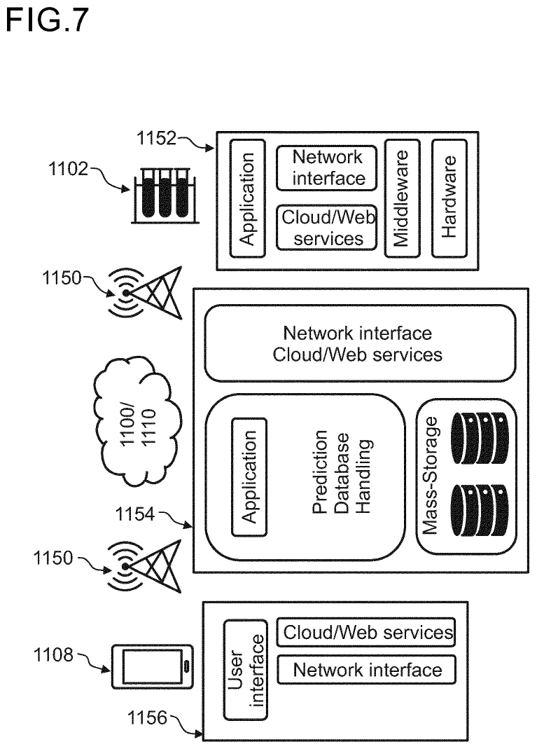

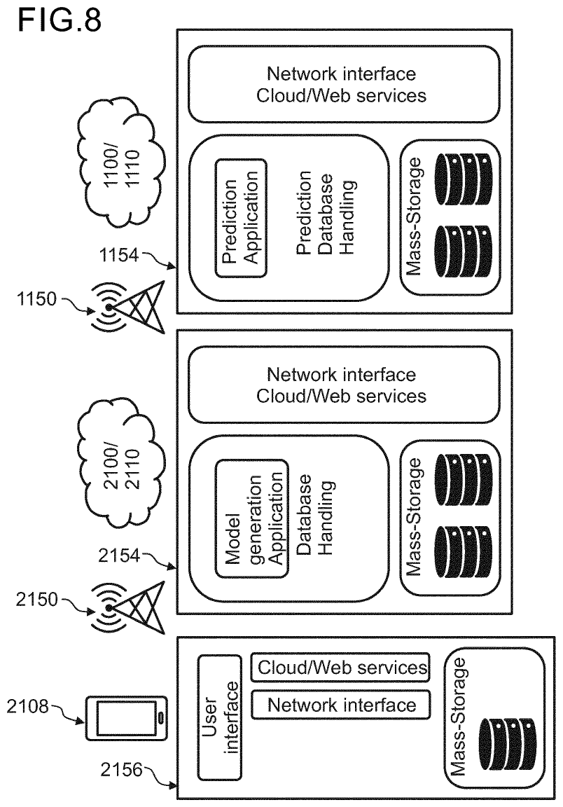

FIG. 7 illustrates a block diagram of an exemplary system architecture of an automated laboratory system 1000 for synthesizing a polymer with a laboratory equipment control device 1102, a network 1150 and the synthesis specification, i.e. recipe, module 1100/1110, and a client device 1108. The automated laboratory system includes a laboratory equipment control device layer 1152 as part of the laboratory equipment control device 1102 as well as a synthesis specification module layer 1154 associated with the synthesis specification module and a remote control or client layer 1156 associated with the client device 1108. The laboratory equipment control device layer can be split into several hierarchical layers: the hardware, the middleware and the interface layer. The hardware layer relates to hardware resources such as sensors and actuators, in particular for controlling synthesis of a polymer. The middleware relates to any of the known middleware for laboratory or plant synthesis operations. One example is LABS/QM, providing different abstractions to hardware, network and operating system such as low-level device control and message passing. The communication layer relates to communication protocols one the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The synthesis specification module layer 1154 may include: a mass storage layer, the computing layer, the interface layer. The storage layer is configured to provide mass storage for the data-driven determination model for providing a recipe, i.e. synthesis specification, of a polymer based on an application performance measure, as described in detail above. In particular, the functions performed by the apparatus, as described above, can be provided as program code means stored on the mass storage. Furthermore, synthesis specifications for a plurality of polymers can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on target properties. Such functionalities can include determining based on a target measure and the determination model a digital representation of a target polymer, generating a synthesis specification from the digital representation of the target polymer, and providing the synthesis specification as control data to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the synthesis specification module layer 1154 or the laboratory equipment control device layer 1152. Users may be provided with a UI for selecting a target measure and an application condition for the target measure, the target measure may also comprise a range of application performance measure values. In other examples, the users may be provided with a UI for selecting more than one target measure and respective values. The applications may be configured for users to monitor and control the laboratory equipment control device and the operation remotely. In other examples, the client device layer and the synthesis specification module layer may be integrated into one device. The alternatives described here are only for illustration purposes and should not be considered limiting.

FIG. 7 illustrates a block diagram of an exemplarily system architecture of a system and apparatus for generating a determination model for determining an application performance measure, a network 2150 and a model generating module 2100/2110 that can be regarded as or comprising a training model apparatus, a synthesis specification module 1100/1110, and a client device 2108. The system for generating a determination model includes a model generating module layer 2154 as part of model generating module and a client layer 2156 associated with the client devices 2108.

The model generating module layer 2154 may include: a mass storage layer, a computing layer, an interface layer. The storage layer is configured to provide mass storage for the data-driven determination model as described above. Furthermore, the mass storage is configured for storing synthesis specifications for polymers and measured biodegradabilities for one or more habitats. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a determination model for determining an application performance measure of a polymer. Such functionalities may include receiving for at least two previously measured polymers their respective digital representations associated with a synthesis specification, measurement data of at least one application performance measure in at least one habitat for each of the at least two previously measured polymers, receiving at the model generating module the digital representation of at least one unmeasured polymer, training the model according to the above described training principles based on the digital representation of the at least two previously measured polymers, the measurement data of the application performance measure in at least one application condition for each of the at least two previously measured polymers, and, preferably, a similarity measure between the digital representation associated with the synthesis specification of each of the at least two previously measured polymers and the respective digital representation associated with a synthesis specification of the at least one unmeasured polymer, and providing via an output interface the determination model for the application performance measure. The model generating module layer may be configured for deploying the generated model and the synthesis specification database to the synthesis specification module layer. This may include storing the generated model and the synthesis specification database in the mass storage devices associated with the synthesis specification module.

The model generating module layer may further be configured for determining a digital representation of the polymer associated with the synthesis specification from the synthesis specification. The digital representation may include a set of polymer physicochemical parameters and polymer physicochemical parameter values associated with a synthesis specification of each measured polymer. One way of deriving these polymer physicochemical parameters can be to apply the SMILES algorithm or any other already above described principle. In the case, where the model is generated based on the digital representation derived from the recipe, a relation between the synthesis specification and the physicochemical parameters may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain synthesis specifications for polymers, and for at least two polymers at least one application performance measure. The client layer further provides an interface for end-users. For end-users, the client layer 2156 may run client side Web applications, which provide interfaces to the model generation module layer 2154 or the mass storage devices associated with the client layer. Users may be provided with a UI for selecting a test method as application condition for which the application performance measure shall be determined. The user may further be provided with a UI for selection of the synthesis specification data. The user interface may also provide an option for uploading the selected data to the model generating module layer and optionally an option to initiate model generation.

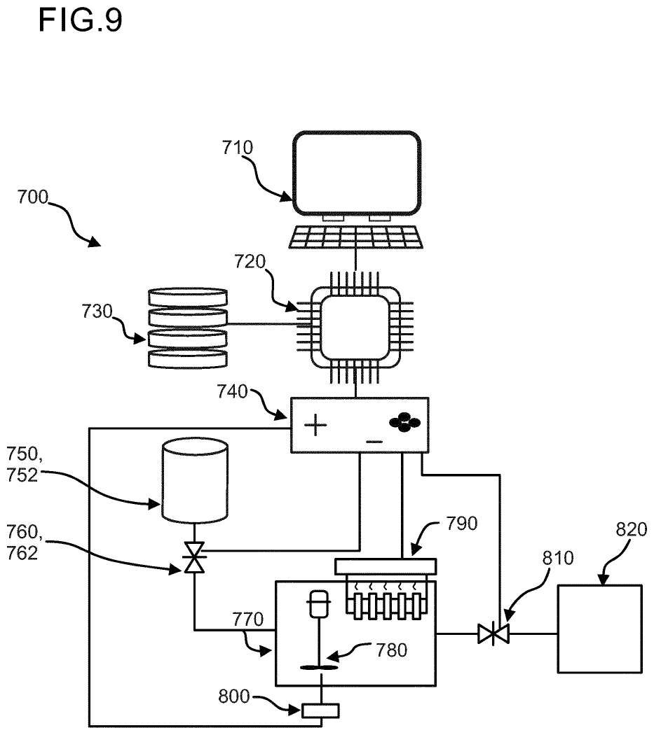

FIG. 9 shows an exemplary system 700 for producing a chemical product based on a synthesis specification generated according to the invention. In this example the system comprises a user interface 710 and a processor 720, associated with a control unit 740. The user interface 710 and the processor 720 can be associated with or realized in accordance with the principles described above, in particular, can be adapted to perform a computer implemented method to determine a target polymer and/or synthesis specification based on a determined application performance measure, as described above. The control unit 740 is, for example, configured for receiving control data generated according to the invention as described above, in particular, to receiving control data generated based on a synthesis specification of a polymer comprising a target measure. In this example the control data is provided from a data base 730, in other examples, however the control data can also be provided from a server or any other computational unit for distributing data. Vessels 750, 752 each contain a component of the chemical product, for example, pre-polymers, catalysts, etc. In general more than two vessels are present, however, in this example for illustrative purposes only two are shown. Valves 760, 762 are associated with vessels 750, 752. Valves 750 and 752 can be controlled to dose appropriate amounts of each component into reactor 770, according to the synthesis specification. A motor 800 of a mixer 780 may also be controlled by the control unit according to the synthesis specification. An optional heater 790 may also be controlled according to the synthesis specification. Finally, an exit valve 810 in fluid communication with the reactor may be controlled by the control unit to provide the chemical product to a container or test system 820.

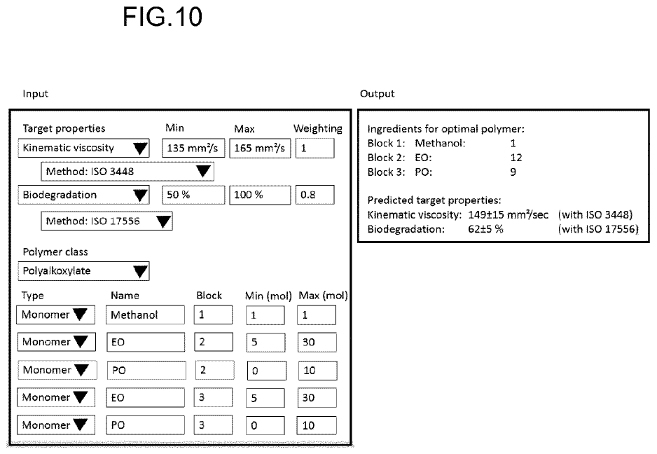

FIG. 10 shows exemplarily and schematically a possible user interface for interacting, for example, with a processor performing the above described method for determining a target polymer with a target measure, being in this example, a target biodegradability. In this example, an input screen is shown on the left. The input screen allows for a definition of a target biodegradability, for instance, in form of minimal and maximal values. In this example, a target biodegradability refers to a determination of the ultimate aerobic biodegradability of a polymer in soil by measuring the oxygen demand in a respirometer or the amount of carbon dioxide evolved within 2 years according to ISO 17556 standard. Optionally, the input can also refer to another target application property, in this example, a target kinematic viscosity and in a weighting of the two targets. Further, the input screen can allow to provide constrains for the target polymer, for example, as shown, constraints to a polymer class that can also be selected via a drop down menu. Here as exemplary case the polymer is constrained to the class of polyalkoxylates. Moreover, additional constrains for the polymer can be provided as shown in FIG. 10. However, this input option can also be omitted and as described above a general synthesis specifications can be provided by a database or can be generated by known methods. Moreover, the input screen can also allow to provide additional or other constrains with respect to the target polymer or the target synthesis specification. Further, the input screen can allow to select a respective habitat and optionally also habitat descriptor values. Here in this example the habitat is determined based on the measurement standard selected for the biodegradability indicating that the habitat refers to soil. Generally, the input can also refer to further information, for example, to defining habitat descriptors, intended applications, measurement method, etc. For this example, it can be shown that a good prediction accuracy can be achieved when utilizing as polymer types a descriptor referring to a molar weight of the polymer, a descriptor referring to an amount of subgroups and chemical motifs, and a descriptor referring to a hydrophiliy of the polymer. The values for such polymer descriptors can then be determined in accordance with the above described principles for a starting polymer and also for respective amended polymers until a target polymer can be found. An exemplary output screen is shown on the right of FIG. 10. In this example, the output screen provides a target polymer that fulfills the target biodegradability and also the target kinematic viscosity. Moreover, the respective components of the polymer are provided in form of component types and associated component, amount and block. Optionally, also further information on the determined target polymer could be provided, for example, an associated synthesis specification.

In the following some examples for the application of the invention to determine a biodegradability are presented. In particular, in these examples the model is trained to determine a biodegradability of a polymer in a specific habitat. For each example, the biodegradation model was trained for a specific class of polymers with a training data set comprising between 50 and 100 different polymers in the polymer class and respective biodegradations in a respective habitat. Moreover, each model was trained to determine a biodegradability as percentage of polymer material converted to CO₂ after 28 days based on the calculated theoretical oxygen demand of the polymer material assuming a total carbon conversion. Further, the model utilized for polyalkoxylates in waste water was trained in accordance with the standard OECD301-Test and the model utilized for polyesters in soil was trained in accordance with ISO 17556 for better comparability.

In a first example, the biodegradation model has been trained for polyalkoxylates in waste water and was based on a two-step approach in which in a first step a Random Forest model was trained and used to select polymer descriptors that are then utilized in a second step in a trained Linear Regression model for determining the biodegradability. The trained biodegradation model, in this case, the linear Regression Model, has then been applied to Plurafac LF 221 utilizing respective physicochemical characteristics. The output of the biodegradation model resulted in a biodegradation of 91%, meaning that 91% of the polymer material has been converted to CO₂ based on the calculated theoretical oxygen demand of the polymer material assuming a total carbon conversion, wherein a measured biodegradation for this polymer is reported as lying above 60%.

In a second example, the biodegradation model has been trained for polyalkoxylates in waste water in the same way as in the first example. In particular, for both exampled the same biodegradation model can be used. The biodegradation model has then been applied to Pluriol E 200 utilizing respective physicochemical characteristics. The output of the biodegradation model resulted in a biodegradation of 96%, meaning that 96% of the polymer material has been converted to CO₂ based on the calculated theoretical oxygen demand of the polymer material assuming a total carbon conversion, wherein a measured biodegradation for this polymer is reported as lying above 70%.

In a third example, the biodegradation model has been trained for amine terminated polyalkoxylates in waste water in the same way as in the first example. In particular, for both exampled the same biodegradation model can be used. The biodegradation model has then been applied to Jeffamine D 230 utilizing respective physicochemical characteristics. The output of the biodegradation model resulted in a biodegradation of 7%, meaning that 7% of the polymer material has been converted to CO₂ based on the calculated theoretical oxygen demand of the polymer material assuming a total carbon conversion, wherein a measured biodegradation for this polymer is reported at 7.2%.

In a fourth example, the biodegradation model has been trained for polyester in soil. In this example, the trained biodegradation model is based on a trained partial least squares model. The biodegradation model has then been applied to Lupraphen 1619/1 utilizing respective physicochemical characteristics. The output of the biodegradation model resulted in a biodegradation of 49%, meaning that 49% of the polymer material has been converted to CO₂ based on the calculated theoretical oxygen demand of the polymer material assuming a total carbon conversion, wherein a measured biodegradation for this polymer is reported as lying above 60%.

With an expected measurement error for the measured values of +/−10% for waste water and +/−10% for soil, the results of the trained biodegradation models lie within a suitable accuracy range for the intended application. Thus, a training using a training data set with only 50 polymer data points already provides a suitable accuracy. Utilizing a data set with more polymers might even lead to a higher accuracy. A biodegradation model can thus be trained accordingly depending on an accuracy suitable for a respective application.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the digital representation and the determination model, the determining of the application performance measure, the providing of the application performance measure, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for determining a performance measure. A providing unit provides a digital representation of a target polymer indicative of characterising parameters of a polymer. A providing unit provides a digital representation of application conditions indicative of application parameters defining the application conditions. A providing unit provides a model based on the provided digital representation of the application conditions. The model is adapted to determine a performance measure of a polymer with respect to the application conditions. The model is a data driven model parameterised with respect to the application conditions such that it can determine a performance measure of a polymer based on the digital representation of the polymer. A determining unit for determining the performance measure of the target polymer based on the provided determination model and the digital representation of the target polymer.

1. A computer implemented method for determining a measure for an application performance of a target polymer, wherein the method comprises:
providing a digital representation of a target polymer indicative or associated with characterising parameters of a polymer,
providing a digital representation of application conditions for the target polymer, wherein a digital representation of the application conditions is indicative or associated with application parameters defining the application conditions,
providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, and
determining the application performance measure of a target polymer based on the provided determination model and the digital representation of the target polymer.

2. The method according to claim 1, wherein the digital representation of the application conditions refers to a representation of a predetermined norm and/or testing procedure with respect to the application performance measure and the application parameters define the predetermined norm and/or testing procedure.

3. The method according to claim 1, wherein the application conditions refer to anyone of a biodegradation habitat, as standardised testing procedure for burning behaviour, a product application environment, and a fluid environment, and the application performance measure refers respectively to any of a biodegradability, a burning behaviour, an adhesion property, a mechanical property, and a solubility.

4. The method according to claim 1, wherein the determination model is parameterised to further determine the application performance measure based on the application parameters such that the application performance measure is determined based on the provided determination model, the digital representation of the polymer and further the application parameters.

5. A computer implemented method for determining a target synthesis specification indicative of a target polymer comprising a target measure for an application performance, wherein the method comprises:
providing a target measure for a target application performance of a polymer,
providing a digital representation of a potential target synthesis specification indicative or associated with a potential target polymer,
providing a digital representation of application conditions, wherein the digital representation is indicative of application parameters defining the application conditions,
providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer,
determining the application performance measure of the potential target polymer based on the provided determination model and the digital representation of the potential target synthesis specification, and
comparing the determined application performance measure of the potential target polymer with the target measure and, based on the comparison, either i) determining the potential target polymer as the target polymer and the potential target synthesis specification as the target synthesis specification, or ii) providing a new potential target synthesis specification of a new potential target polymer and repeating the determination of the application performance measure utilizing the new potential target synthesis specification of the new potential target polymer.

6. The method according to claim 5, further comprising:
providing a target technical application property for the target polymer and providing the potential target synthesis specification based on the provided target technical application property such that the potential target polymer fulfils the provided target technical application property.

7. The method according to claim 6, wherein the providing of a new potential target synthesis specification is based on amending the provided target application property and providing the new potential target synthesis specification such that the potential target polymer fulfils the amended target application property.

8. The method according to claim 6, wherein the providing of the potential target synthesis specification based on the provided target technical application property comprises utilizing an additional determination model adapted to determine a technical application property of a polymer based on the digital representation of a polymer, wherein the additional determination model is a data driven model parameterised such that the determines based on the digital representation of the polymer the technical application property associated with a polymer.

9. The method according to claim 5, wherein the providing of the target synthesis specification of the target polymer comprises providing control signals adapted for controlling an industrial plant for producing the target polymer in accordance with a target synthesis specification.

10. An interface method for providing an interface, wherein the interface method comprises:
receiving as input a digital representation and an application condition via a user interface and providing the received digital representation and the application condition to a processor performing the method according to claim 1, and
providing the determined application performance measure of the polymer or the determines synthesis specification, respectively, to a user via a user interface as result, wherein the result is received from the processor performing the method according to claim 1.

11. A computer implemented training method for training a data driven based determination model for parameterizing the determination model, wherein the training method comprises:
providing training data associated with a predetermined application condition, wherein the training data comprises a) digital representations of a plurality of training polymers indicative of characterizing parameters for each of the training polymers, and b) an application performance measure for the respective application condition associated with each training polymer,
providing a data driven based trainable determination model,
training the provided data driven based determination model based on the provided training data such that the trained determination model is adapted to determine an application performance measure of a polymer based on a digital representation of the polymer, in particular, based on the characterizing parameters, preferably, polymer physicochemical parameters, more preferably, polymer descriptors, indicated by the digital representation, and
providing the trained determination model.

12. An apparatus for determining a measure for an application performance of a target polymer, wherein the apparatus comprises:
a digital representation providing unit for providing a digital representation of a target polymer indicative or associated with characterising parameters of a polymer,
an application conditions providing unit for providing a digital representation of application conditions for the target polymer, wherein a digital representation of the application conditions is indicative or associated with application parameters defining the application conditions,
a model providing unit for providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer, and
a determining unit for determining the application performance measure of a target polymer based on the provided determination model and the digital representation of the target polymer.

13. An apparatus for determining a target synthesis specification indicative of a target polymer comprising a target measure for an application performance, wherein the apparatus comprises:
a target providing unit for providing a target measure for a target application performance of a polymer,
a digital representation providing unit for providing a digital representation of a potential target synthesis specification indicative or associated with a potential target polymer,
an application conditions providing unit providing a digital representation of application conditions, wherein the digital representation is indicative of application parameters defining the application conditions,
a model providing unit for providing a determination model based on the provided digital representation of the application conditions, wherein the determination model is adapted to determine an application performance measure of a polymer with respect to the application conditions, wherein the determination model is a data driven model parameterised with respect to the application conditions such that it can determine an application performance measure of a polymer based on the digital representation of the polymer,
a determining unit for determining the application performance measure of the potential target polymer based on the provided determination model and the digital representation of the potential target synthesis specification, and
an iteration control unit (116) for comparing the determined application performance measure of the potential target polymer with the target measure and, based on the comparison, either i) determining the potential target polymer as the target polymer and the potential target synthesis specification as the target synthesis specification, or ii) providing a new potential target synthesis specification of a new potential target polymer and repeating the determination of the application performance measure utilizing the new potential target synthesis specification of the new potential target polymer.

14. A computer program product for determining an application performance measure for a predetermined polymer, wherein the computer program product comprises program code means for causing the apparatus according to claim 12 to execute the method according to claim 1.

15. A computer program product for determining a target synthesis specification, wherein the computer program product comprises program code means for causing the apparatus according to claim 13 to execute the method according to claim 5.
